(19) United States
(12) Patent Application Publication
(10) Pub. No.: US 2018/0291382 A1
(43) Pub. Date: Oct. 11, 2018

(54) Methods for Autocatalytic Genome Editing and Neutralizing Autocatalytic Genome Editing

(71) Applicant: The Regents of the University of California, Oakland, CA (US)

(72) Inventors: Ethan Bier, San Diego, CA (US); Valentino Gantz, La Jolla, CA (US)

(21) Appl. No.: 15/524,440

(22) PCT Filed: Nov. 4, 2015
(86) PCT No.: PCT/US15/58961

§ 371 Date: May 4, 2017

Related U.S. Application Data
(60) Provisional application No. 62/075,534, filed on Nov. 5, 2014.
(60) Provisional application No. 62/101,443, filed on Jan. 9, 2015.

(51) Int. Cl. C12N 15/63; C12N 15/10; C12N 9/22; C12N 15/11; C12N 15/82

(52) U.S. Cl. CPC C12N 15/63; C12N 15/102; C12N 2310/20; C12N 15/11; C12N 15/8279; C12N 9/22

(57) ABSTRACT

The invention provides methods for autocatalytic genome editing based on genomic integration of a construct containing multiple elements. More specifically, the invention provides a method for autocatalytic genome editing or for deleting or neutralizing autocatalytic genome editing based on the CRISPR/Cas9 system, and methods of use thereof, in animals, humans, and plants for eliminating pathogens, targeting suppression of crop pests, strategies to combat virus (e.g., HIV) and other diseases (e.g., cancer) caused by retrovirus, as well as to generate, and reverse, homozygous mutations that are transmitted to nearly all offspring.

### CROSS-REFERENCE TO RELATED APPLICATIONS

This application claims the priority benefit of U.S. provisional application Ser. Nos. 62/075,534 and 62/101,443, filed Nov. 5, 2014 and Jan. 9, 2015, respectively. The entire contents of which are hereby incorporated by reference herein.

### STATEMENT AS TO FEDERALLY SPONSORED RESEARCH

This invention was made with government support under Grant Nos. AI070654 and NS029870 awarded by the National Institutes of Health. The government has certain rights in the invention.

### BACKGROUND OF THE INVENTION

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced bacteria genomes and 90% of sequenced archaea. CRISPRs are often associated with Cas or similar genes that code for endonucleases related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. Improved methods and compositions for use in eukaryotic cells and organisms are needed for improved genomic engineering technologies.

### SUMMARY OF THE INVENTION

The present invention discloses methods and compositions for selectively introducing and/or neutralizing the spread of Mutagenic Chain Reaction (MCR) elements from organisms carrying them that do not affect organisms lacking such elements.

MCR for autocatalytic genome editing is based on genomic integration of an MCR construct containing multiple elements. In some embodiments, the MCR invention either: a) injects the MCR construct as a DNA plasmid into the germline of an organism and obtains transgenic organisms carrying this insertion on one copy of a chromosome from which it can spread to the other chromosome (creating potential homozygous mutations) as well as propagating the same mutation via the germline to nearly all its offspring, or b) introduces the MCR construct into somatic cells in an organism (e.g., using a plasmid or viral expression vector) such that the construct spreads to other cells within that organism. Therefore, the MCR provides an autocatalytic method to generate homozygous mutations that propagate with high fidelity via the germline to nearly all progeny who themselves become homozygous for the mutation.

In another aspect, the present invention also provides for selective deletion and/or neutralization of MCR elements, in a system referred to as the Neutralizing Chain Reaction (NCR). NCR elements, which are also known as Elements for Reversing the Autocatalytic Chain Reaction (ERACRs), can be comprised of a number of elements whereby to inject the construct as a DNA plasmid together with a plasmid source of Cas9 protein into the germline of an organism and obtain transgenic organisms carrying this insertion. Organisms carrying this construct would then be crossed to MCR individuals (or released into an environment containing MCR individuals) whereupon NCR would act on the MCR chromosome to delete the MCR element and could also restore function of the host locus via a recoded transgene.

The present inventions are based on a well-known bacterial immunity function known as the CRISPR/Cas9 system that is based on two components. The first component is an endonuclease such as Cas9, that has a binding site for the second component, which is the guide polynucleotide (e.g., guide RNA). The guide polynucleotide (e.g., guide RNA) directs the endonuclease (e.g., Cas9) protein to DNA templates (e.g., a bacteriophage integrated into the bacterial chromosome) based on sequence homology. The Cas9 protein then cleaves that template leading to secondary mutations during DNA repair. The CRISPR/Cas system has been used for gene editing (e.g., adding, disrupting or changing the sequence of specific genes) and gene regulation in many species. By delivering the Cas9 protein and appropriate guide polynucleotides (e.g., guide RNAs) into a cell, the organism's genome can be cut at a desired location. This system has recently been found to be adaptable to many organisms including mammalian cells, fruit flies, and plants. The broad adaptability of this system has led to significant strides in refining this system and the generation of many applications. The present invention may be applied to flies, mosquitoes, human cells, and plants, for example. The present invention provides methods and constructs for generating and neutralizing homozygous germline transmissible mutations.

The invention provides in certain embodiments, a method for autocatalytic genome editing comprises genomically integrating a construct comprising four elements: (1) a gene encoding an endonuclease (e.g., Cas9 protein), (2) a sequence encoding one or more guide polynucleotides (e.g., guide RNAs), (3) an effector cassette, and (4) homology arms flanking the above three transgenes that target insertion of those elements (1-3) into the genome at the site determined by the sequence flanking the guide polynucleotide(s) (e.g., guide RNA(s)) (element 2).

In certain embodiments, the guide polynucleotide (e.g., guide RNA) once expressed binds to Cas9 protein and directs sited directed cleavage of the genome at a specific site.

In certain embodiments, the sequence encoding one or more guide polynucleotides is under a control of a separate promoter. In certain embodiments, the separate promoter is an RNA-polymerase-I or III promoter.

In certain embodiments, the construct is injected as a DNA plasmid into a germline of an organism to obtain a transgenic organism.

In certain embodiments, homozygous mutations are created wherein the transgenic organism carrying the inserted construct on one copy of a chromosome from which it spreads to another chromosome.

In certain embodiments, germline transmissible mutations are created wherein the transgenic organism carrying the inserted construct is propagated with high fidelity via the germline to nearly all offspring.

In certain embodiments, the construct is introduced into somatic cells in an organism so that the construct can be spread to other cells within that organism.

In certain embodiments, the construct is introduced using a plasmid or viral expression vector.

In certain embodiments, the autocatalytic genome editing is applied to animals, humans, or plants.

In certain embodiments, the autocatalytic genome editing is used to eliminate pathogens. In certain embodiments, the pathogen is a malaria causing parasite (e.g., Plasmodium falciparum).

In certain embodiments, the autocatalytic genome editing is used to target suppression of crop pests to those actively attaching a crop of interest.

In certain embodiments, the autocatalytic genome editing is used to combat viruses or retroviruses or other diseases independent of the type and stage of disease progression. In certain embodiments, the virus is HIV. In certain embodiments, the disease is cancer.

In certain embodiments, the autocatalytic genome editing generates scoreable recessive mutant phenotypes in a single generation.

The invention provides in other embodiments, a construct for autocatalytic genome editing comprising four elements:
(1) a gene encoding a Cas9 protein,
(2) a sequence encoding one (or multiple) guide polynucleotide (e.g., guide RNA) sequence under a control of a separate promoter,
(3) an effector cassette, and
(4) homology arms flanking the above three transgenes that target insertion of those elements (1-3) into the genome at the site determined by the sequence flanking the guide polynucleotide(s) (e.g., guide RNA(s)) (element 2).

Accordingly, one aspect of the invention provides a method of neutralizing a mutagenic chain reaction (MCR) element in a cell or organism, the method comprising genomically integrating a neutralizing chain reaction (NCR) element from an NCR construct into the cell or organism, wherein:

the MCR element comprises:

(a) at least one sequence encoding at least one guide polynucleotide, wherein the at least one sequence encoding at least one guide polynucleotide is genomically integrated in the cell or organism; and

(b) a gene encoding an endonuclease;

the NCR element comprises:

(a) at least one sequence encoding at least one guide polynucleotide directing cleavage within or on both sides of the MCR element; and

(b) at least one sequence encoding at least two guide polynucleotides directing cleavage within or outside of the MCR element or no gene encoding an endonuclease; and the NCR construct comprises:

(a) the NCR element; and

(b) homology arms flanking the at least one guide polynucleotide that directly abut the endonuclease cut site(s) determined by the at least one guide polynucleotide.

In some embodiments of any one of the methods or constructs described herein, the guide polynucleotides are guide RNAs.

In some embodiments of any one of the methods or constructs described herein, the endonuclease is a Cas protein, such as Cas9.

In some embodiments of any one of the methods or constructs described herein, the cell or organism is a cell. In some embodiments of any one of the methods or constructs described herein, the cell or organism is an organism.

In some embodiments of any one of the methods or constructs described herein, the genomically integrating comprises genomically integrating into a chromosome of the cell or organism.

In some embodiments of any one of the methods or constructs described herein, the gene encoding an endonuclease is genomically integrated in the cell or organism. In some embodiments of any one of the methods or constructs described herein, the gene encoding an endonuclease is not genomically integrated in the cell. In some embodiments of any one of the methods or constructs described herein, the gene encoding an endonuclease is located on a plasmid or artificial chromosome.

In some embodiments of any one of the methods or constructs described herein, the at least one sequence encoding at least one guide polynucleotide in the MCR element is genomically integrated in the cell or organism.

In some embodiments of any one of the methods or constructs described herein, the MCR element is genomically integrated in the cell or organism.

In some embodiments of any one of the methods described herein, the method further comprises deletion of the gene encoding the endonuclease from the genome. In some embodiments of any one of the methods described herein, the method further comprises deletion of the at least one sequence encoding at least one guide polynucleotide in the MCR element from the genome. In some embodiments of any one of the methods described herein, the method further comprises deletion of the MCR element from the genome. In some embodiments of any one of the methods described herein, the method further comprises disruption of the gene encoding the endonuclease. In some embodiments of any one of the methods or constructs described herein, the disruption of the gene encoding the endonuclease in the genome comprises a deletion, insertion, or mutation of at least one amino acid of the endonuclease.

In some embodiments of any one of the methods or constructs described herein, the directing cleavage within or on both sides of the MCR element comprises directing cleavage on the same allele as the MCR element.

In some embodiments of any one of the methods or constructs described herein, the NCR construct does not comprise a gene encoding an endonuclease. In some embodiments of any one of the methods or constructs described herein, the NCR element does not comprise a gene encoding an endonuclease.

In some embodiments of any one of the methods or constructs described herein, the at least one sequence encoding at least one guide polynucleotide in the NCR element comprises a different sequence than the at least one sequence encoding at least one guide polynucleotide in the MCR element.

In some embodiments of any one of the methods or constructs described herein, the at least one sequence encoding at least one guide polynucleotide in the NCR element directs cleavage within the MCR element. In some embodiments of any one of the methods or constructs described herein, the at least one sequence encoding at least one guide polynucleotide in the NCR element directs cleavage within the gene encoding the endonuclease. In some embodiments of any one of the methods or constructs described herein, the at least one sequence encoding at least one guide polynucleotide in the NCR element directs cleavage within the at least one sequence encoding at least one guide polynucleotide in the MCR element.

In some embodiments of any one of the methods or constructs described herein, the at least one sequence encoding at least one guide polynucleotide in the NCR element directs cleavage on both sides of the MCR element. In some embodiments of any one of the methods or constructs described herein, the at least one sequence encoding at least one guide polynucleotide in the NCR element directs cleavage on both sides of the gene encoding the endonuclease. In some embodiments of any one of the methods or constructs described herein, the at least one sequence encoding at least one guide polynucleotide in the NCR element directs cleavage on both sides of the at least one sequence encoding at least one guide polynucleotide in the MCR element.

In some embodiments of any one of the methods or constructs described herein, the NCR construct and/or NCR element comprises one guide polynucleotide. In some embodiments of any one of the methods or constructs described herein, the one guide polynucleotide directs one cleavage site. In some embodiments of any one of the methods or constructs described herein, the one guide polynucleotide directs cleavage within the MCR element. In some embodiments of any one of the methods or constructs described herein, the one guide polynucleotide directs cleavage within the gene encoding the endonuclease. In some embodiments of any one of the methods or constructs described herein, the one guide polynucleotide directs cleavage within the at least one sequence encoding at least one guide polynucleotide in the MCR element. In some embodiments of any one of the methods or constructs described herein, the one guide polynucleotide directs two cleavage sites. In some embodiments of any one of the methods or constructs described herein, the one guide polynucleotide directs cleavage on both sides of the endonuclease. In some embodiments of any one of the methods or constructs described herein, the one guide polynucleotide directs cleavage on both sides of the at least one sequence encoding at least one guide polynucleotide in the MCR element. In some embodiments of any one of the methods or constructs described herein, the one guide polynucleotide directs cleavage on both sides of the MCR element.

In some embodiments of any one of the methods or constructs described herein, the NCR construct and/or NCR element comprises two guide polynucleotides. In some embodiments of any one of the methods or constructs described herein, the two guide polynucleotides direct two cleavage sites. In some embodiments of any one of the methods or constructs described herein, the two guide polynucleotides direct cleavage within the MCR element. In some embodiments of any one of the methods or constructs described herein, the two guide polynucleotides direct cleavage within the gene encoding the endonuclease. In some embodiments of any one of the methods or constructs described herein, the two guide polynucleotides direct cleavage within the at least one sequence encoding at least one guide polynucleotide in the MCR element. In some embodiments of any one of the methods or constructs described herein, the two guide polynucleotides direct cleavage on both sides of the gene encoding the endonuclease. In some embodiments of any one of the methods or constructs described herein, the two guide polynucleotides direct cleavage on both sides of the at least one sequence encoding at least one guide polynucleotide in the MCR element. In some embodiments of any one of the methods or constructs described herein, the two guide polynucleotides direct cleavage on both sides of the MCR element.

In some embodiments of any one of the methods or constructs described herein, the at least one sequence encoding at least two guide polynucleotides in the NCR element comprises at least two sequences encoding at least two guide polynucleotides.

In some embodiments of any one of the methods or constructs described herein, the NCR element is genomically integrated using homology directed repair. In some embodiments of any one of the methods or constructs described herein, the NCR element is not genomically integrated using non-homologous end joining. In some embodiments of any one of the methods or constructs described herein, the NCR element is genomically integrated with an efficiency of at least 25%, at least 50%, at least 75%, at least 80%, at least 85%, at least 90%, or at least 95%.

In some embodiments of any one of the methods or constructs described herein, the NCR construct is located on a plasmid. In some embodiments of any one of the methods or constructs described herein, the NCR construct is located on a chromosome. In some embodiments of any one of the methods or constructs described herein, the homology arms in the NCR construct are located on a plasmid. In some embodiments of any one of the methods or constructs described herein, the homology arms in the NCR construct are located on a chromosome. In some embodiments of any one of the methods or constructs described herein, the homology arms in the NCR construct are at least 25 nucleotides, at least 50 nucleotides, at least 75 nucleotides, at least 100 nucleotides, or at least 200 nucleotides in length.

In some embodiments of any one of the methods or constructs described herein, the MCR element is located on a first copy of a chromosome and the NCR element is located on a second copy of a chromosome.

In some embodiments of any one of the methods or constructs described herein, the NCR element further comprises a corrected recoded gene or cis-regulatory element that is not cut by the at least one guide polynucleotide in the MCR element. In some embodiments of any one of the methods or constructs described herein, the NCR element further comprises a corrected effector cassette.

In some embodiments of any one of the methods described herein, the method further comprises restoring a genetic function of a locus mutated by the MCR element.

In some embodiments of any one of the methods or constructs described herein, the NCR construct is injected as a DNA plasmid into a germline of an organism to obtain a transgenic organism.

In some embodiments of any one of the methods described herein, the method further comprises generating homozygous mutations in the cell or organism.

In some embodiments of any one of the methods described herein, the method further comprises genomically integrating the NCR element into both copies of a chromosome of the cell or organism.

In some embodiments of any one of the methods described herein, the method further comprises propagating the NCR element via the germline to offspring of the organism.

In some embodiments of any one of the methods or constructs described herein, the NCR construct is introduced into somatic cells in the organism.

In some embodiments of any one of the methods described herein, the method further comprises spreading the NCR element to other cells within the organism.

In some embodiments of any one of the methods or constructs described herein, the NCR construct is injected as a DNA plasmid into a germline or introduced via DNA plasmid or viral expression vector into somatic cells of the organism to obtain transgenic organisms resulting in homozygous or nearly fully converted germline mutations.

In some embodiments of any one of the methods or constructs described herein, the NCR construct is introduced using a plasmid or viral expression vector.

In some embodiments of any one of the methods or constructs described herein, the organism is an animal, human, microorganism, insect, plant, or any combination thereof. In some embodiments of any one of the methods or constructs described herein, the organism is a model organism. In some embodiments of any one of the methods or constructs described herein, the organism is a virus, prokaryote, eukaryote, protist, fungus, invertebrate animal, vertebrate animal, microorganism, pathogen, agriculture pest, or any combination thereof. In some embodiments of any one of the methods or constructs described herein, the cell is from a virus, prokaryote, eukaryote, protist, fungus, invertebrate animal, vertebrate animal, microorganism, pathogen, agriculture pest, or any combination thereof.

One aspect of the invention provides a construct for neutralizing autocatalytic genome editing, the construct comprising:
(a) at least one sequence encoding at least one guide polynucleotide directing cleavage within or on both sides of the MCR element;
(b) homology arms flanking the at least one guide polynucleotide that directly abut the endonuclease cut site(s) determined by the at least one guide polynucleotide; and
(c) at least one sequence encoding at least two guide polynucleotides directing cleavage within or outside of the MCR element or no gene encoding an endonuclease;

wherein the MCR element comprises:

(a) at least one sequence encoding at least one guide polynucleotide, wherein the at least one sequence encoding at least one guide polynucleotide is genomically integrated in a cell or organism; and

(b) a gene encoding an endonuclease.

One aspect of the invention provides a method of genomically integrating a neutralizing chain reaction (NCR) element into a cell or organism, the method comprising:

introducing into the cell or organism an NCR construct comprising:

(a) at least one sequence encoding at least one guide polynucleotide directing cleavage within or on both sides of an MCR element;

(b) homology arms flanking the at least one guide polynucleotide that directly abut the endonuclease cut site(s) determined by the at least one guide polynucleotide; and

(c) at least one sequence encoding at least two guide polynucleotides directing cleavage within or outside of the MCR element or no gene encoding an endonuclease; and genomically integrating an NCR element comprising:

(a) at least one sequence encoding at least one guide polynucleotide directing cleavage within or on both sides of the MCR element; and

(b) at least one sequence encoding at least two guide polynucleotides directing cleavage within or outside of the MCR element or no gene encoding an endonuclease;

wherein the MCR element comprises:

(a) at least one sequence encoding at least one guide polynucleotide; and

(b) a gene encoding an endonuclease; and wherein the cell or organism comprises an endonuclease or a gene encoding an endonuclease.

In some embodiments of any one of the methods or constructs described herein, the cell or organism does not comprise the MCR element. In some embodiments of any one of the methods or constructs described herein, the cell or organism comprises the MCR element.

In some embodiments of any one of the methods or constructs described herein, the NCR construct is introduced using a plasmid or viral expression vector.

In some embodiments of any one of the methods or constructs described herein, the NCR construct does not comprise a gene encoding an endonuclease. In some embodiments of any one of the methods or constructs described herein, the NCR element does not comprise a gene encoding an endonuclease.

One aspect of the invention provides a method for autocatalytic genome editing, the method comprising genomically integrating a mutagenic chain reaction (MCR) element from an MCR construct into a cell or organism, wherein:

the MCR element comprises:

(a) a gene encoding an endonuclease,

(b) at least one sequence encoding at least one guide polynucleotide, and

(c) an effector cassette; and

the MCR construct comprises:

(a) the MCR element; and

(b) homology arms flanking the MCR element, wherein the homology arms directly abut the endonuclease cut site(s) determined by the at least one guide polynucleotide.

In some embodiments of any one of the methods or constructs described herein, the homology arms directly abut the endonuclease cut site(s) determined by the at least one guide polynucleotide, or are separated by less than 100, 75, 50, 40, 30, 20, 10, 9, 8, 7, 6, 5, 4, 3, 2, or 1 nucleic acids.

In some embodiments of any one of the methods or constructs described herein, the guide polynucleotide once expressed binds to the endonuclease and directs site directed cleavage of the genome at a specific site.

In some embodiments of any one of the methods or constructs described herein, the sequence encoding at least one polynucleotide is under a control of a separate promoter, such as an RNA-polymerase-I or III promoter.

In some embodiments of any one of the methods or constructs described herein, the MCR construct is injected as a DNA plasmid into a germline of the organism to obtain a transgenic organism.

In some embodiments of any one of the methods or constructs described herein, homozygous mutations are created wherein the transgenic organism carrying the inserted construct on one copy of a chromosome from which it spreads to another chromosome.

In some embodiments the MCR carries gRNAs that are capable of cutting at other chromosomal sites than the MCR insertion site leading either to mutagenesis (i.e., via NHEJ) or editing (via HDR) of those sites. Such genomic alterations would then propagate along with the MCR as it spreads in a population.

In some embodiments of any one of the methods or constructs described herein, mutations are created wherein the transgenic organism carrying the inserted construct is propagated via the germline to offspring.

In some embodiments of any one of the methods or constructs described herein, the MCR construct is introduced into somatic cells in an organism so that the construct can be spread to other cells within that organism.

In some embodiments of any one of the methods or constructs described herein, the MCR construct is introduced using a plasmid or viral expression vector.

In some embodiments of any one of the methods or constructs described herein, the autocatalytic genome editing is used to target a pathogen, such as Plasmodium falciparum.

In some embodiments of any one of the methods or constructs described herein, the autocatalytic genome editing is used to target suppression of crop disease or crop pests to those actively attacking a crop of interest.

In some embodiments of any one of the methods or constructs described herein, the autocatalytic genome editing targets a virus, retrovirus, a fungus, a parasite, a bacteria, a microorganism, or another disease independent of the type and stage of disease progression. In some embodiments of any one of the methods or constructs described herein, the virus is HIV. In some embodiments of any one of the methods or constructs described herein, the disease is cancer, autoimmune disease, or diabetes, for example.

In some embodiments of any one of the methods or constructs described herein, the autocatalytic genome editing generates scoreable recessive mutant phenotypes in a single generation.

One aspect of the invention provides a construct for autocatalytic genome editing, the construct comprising:

(c) a gene encoding an endonuclease,

(d) at least one sequence encoding at least one guide polynucleotide,

(e) an effector cassette, and

(f) homology arms flanking the gene, the at least one sequence, and the cassette, wherein the homology arms directly abut the endonuclease cut site(s) determined by the at least one guide polynucleotide.

In some embodiments of any one of the methods or constructs described herein, the construct is injected as a DNA plasmid into one or more germline cells or introduced via DNA plasmid or viral expression vector into one or more somatic cells of the organism to obtain a transgenic organism. In some embodiments, this resulting in homozygous mutations or mutations passed on to progeny.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1G are a scheme outlining the Mutagenic Chain Reaction (MCR).

FIGS. 2A-2I are an experimental demonstration of MCR in Drosophila.

FIGS. 3A-3D describe some potential applications of MCR.

FIGS. 4A-4G are a scheme outlining the Neutralizing Chain Reaction (NCR).

FIG. 5 shows a comparison of inheritance via traditional Mendelian versus active genetics.

FIG. 6 shows sequence data for MCR-induced mutations.

FIGS. 7A-7B show exemplary data on the ability of a first generation NCR (i.e., ERACR) to inactivate an MCR about 95% of the time, demonstrating that an NCR can use, e.g., Cas9 provided in trans by the MCR to inactivate the MCR. It also provides evidence for double cutting and copying of the NCR.

FIG. 8 shows an illustration demonstrating CHACR; efficient double cutting by a vector carrying two gRNAs targeting deletion of a region in another region of the genome than the MCR.

FIG. 9 shows illustrations of MCR and/or CHACR in which the reaction is used to drive a gene edit along with the MCR. These methods could be used, e.g., in the gene-drive field since the methods allow for a set of fine genetic alterations to follow along with the MCR as it spread through a population fine-tuning its effect and also reducing any negative side effects may otherwise cause.

### DETAILED DESCRIPTION OF THE INVENTION

The invention provides methods and compositions related to the Mutagenic Chain Reaction (MCR) and the Neutralizing Chain Reaction (NCR) (also referred to as Element for Reversing the Autocatalytic Chain Reaction (“ERACR”)). MCR is a genome editing method. In some embodiments MCR comprises autocatalytic genome editing based on genomic integration of a portion of an MCR construct containing multiple elements. In certain embodiments, the MCR construct comprises: 1) a gene encoding a nuclease (e.g., a Cas protein such as the Cas9 protein); 2) one or more sequences encoding one or more guide polynucleotides (e.g., guide RNAs such as sgRNA, gRNA or chiRNA); 3) an effector cassette (e.g., a nucleotide sequence that is an effector) and 4) homology arms (e.g., sequences flanking the nuclease, the guide polynucleotide, and the effector cassette). In some instances, expression of the endonuclease may be regulated (e.g., by a promoter sequence that is inducible). In some instances, the sequence encoding one or more guide polynucleotides is under the control of a separate promoter (e.g., such as an RNA-polymerase-I or -III promoter, such as the U6 RNA pol-III promoter). The guide polynucleotide (e.g., guide RNA) can be designed to bind to the nuclease (e.g., Cas9 protein) and also designed to direct site directed cleavage of a target nucleic acid (e.g., a genomic loci) at one or more specific sites. In some instances, the homology arms directly abut the endonuclease cleavage sites (e.g., at the 5′ and/or 3′ ends). In some instances, the homology arms target insertion of the gene, one or more sequences, and effector cassette into the genome (e.g., via Homology Directed Repair (HDR)) at the precise endonuclease cleavage site(s) determined by the one or more guide polynucleotides (e.g., guide RNA(s)). In some instances, an MCR construct can be carried on an extragenomic nucleic acid. In some instances an MCR construct is in a DNA plasmid. In some instances an MCR construct is in a viral vector, episomal element, or mini-chromosome.

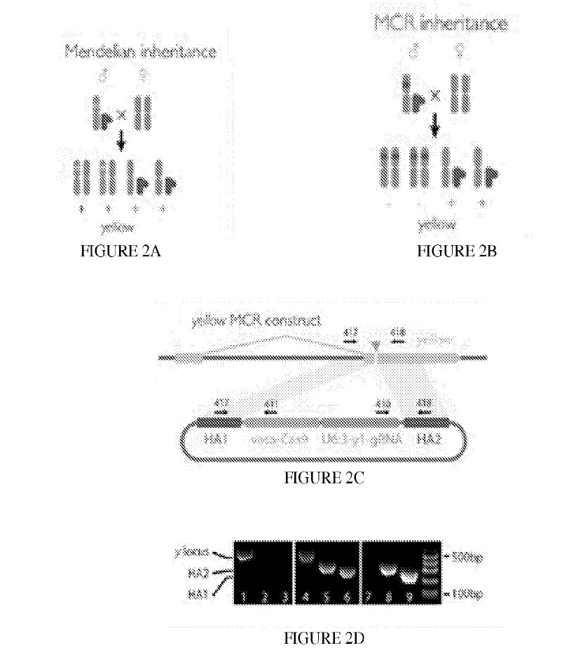

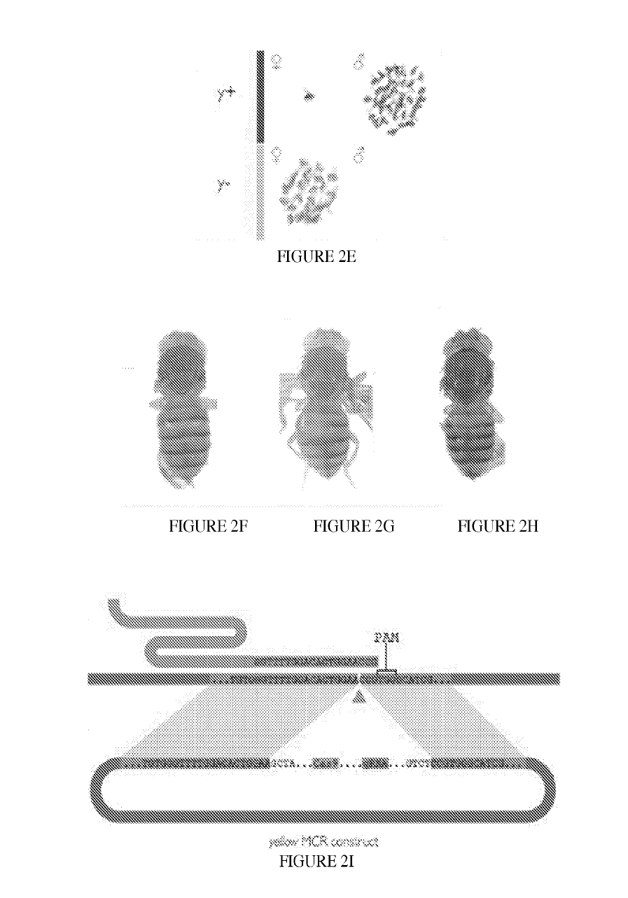

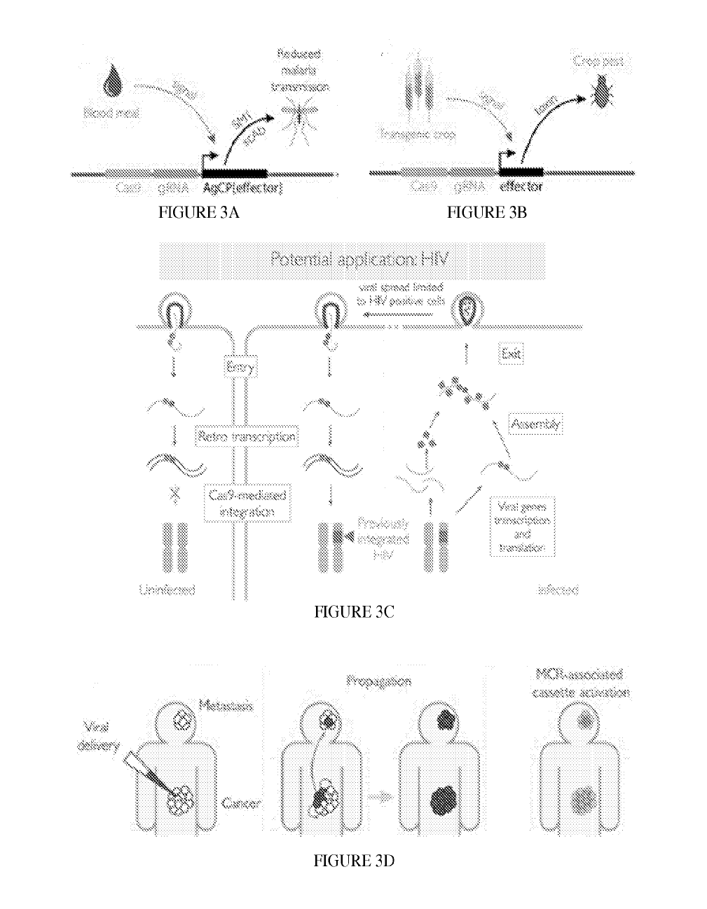

The invention further provides the method of inserting a portion of an MCR construct into the germline of an organism. In some embodiments a transgenetic organism is obtained. In some embodiments the transgenetic organism carries the insertion on one copy of a chromosome. In some embodiments, the insertion on one chromosome can spread to the other chromosome. In some embodiments, the method further comprises generating a homozygous mutation. In some embodiments, the transgenic organism may propagate a mutation via the germline to nearly all of its offspring, as shown in FIGS. 2A-2I. The invention further provides the method of introducing an MCR construct into somatic cells of an organism (e.g., using a plasmid or viral expression vector) such that the construct can spread to other cells within that organism, as shown in FIGS. 3A-3D.

An MCR construct may comprise a polynucleotide (e.g., a single guide RNA). In these cases, the homology arms may directly abut the single cut site, leading to insertion of the MCR element at the cut site (e.g., as shown in FIG. 2I). An MCR construct may comprise two guide polynucleotides (e.g., guide RNAs) that direct cleavage at a certain distance apart. In such cases, the MCR construct comprises flanking homology arms ending precisely at the two cut sites, and the MCR element may lead to deletion of a target sequence (e.g., a target host genome sequence) between the cut sites and insertion of the MCR element within that deletion.

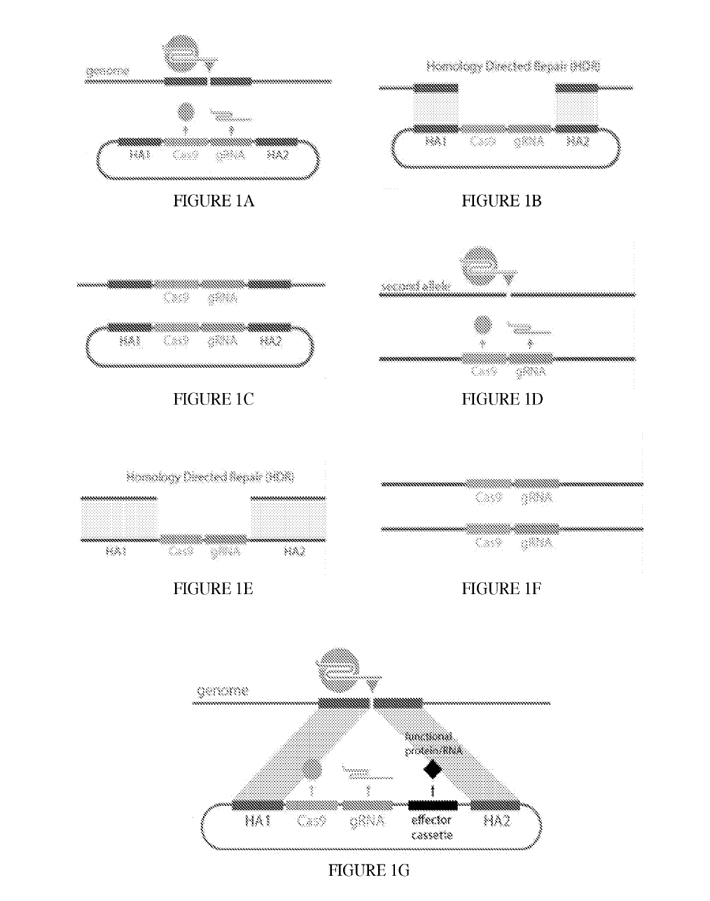

The methods of the disclosure may be targeted to somatic and/or germline cells of an organism. In some embodiments a method may comprise: an injection of the MCR construct as a DNA plasmid into the germline of an organism, e.g., to obtain transgenic organisms carrying this insertion on one copy of a chromosome from which it can spread to the other chromosome (i.e., thereby creating potential homozygous mutations) as well as propagating the mutation via the germline to nearly all of its offspring (see FIGS. 1A-1G, FIGS. 2A-2B). In other embodiments a method may comprise introducing the MCR construct into somatic cells in an organism (e.g., using a plasmid or viral expression vector) such that the construct would spread to other cells within that organism (see FIGS. 3A-3D).

An MCR construct may be integrated into a defined site on a single copy of a chromosome. For instance, specific targeting via the guide polynucleotide (e.g., guide RNA) may direct the endonuclease (e.g., Cas9) to cleave the genome at a specific site, and the MCR construct may be inserted into the site by homologous repair using the homology arms as a template. An MCR insertion event may take place in a germline cell or a somatic cell. By carrying the elements necessary for insertion into the same site on a second copy of the chromosome, the MCR element may cleave the other allele in a cell at the same place and insert itself into the second copy of the chromosome thereby resulting in the insertion becoming homozygous. The MCR insertion may become homozygous in the germline, resulting in progeny of an individual carrying an MCR allele inheriting it. The mutation may spread from a single chromosome to both chromosomes in the next generation to once again become homozygous. In one non-limiting example, FIGS. 2A-2I demonstrate a proof-of-principle example whereby MCR-directed mutagenesis of the Drosophila yellow gene locus, resulted in more than 95% of tested somatic and germline cells being homozygous. In some embodiments, MCR mutations may be homozygous and spread via the germline to nearly all offspring. In some embodiments, MCR mutations can be an efficient way to spread genomic material through a population via the germline. In some embodiments, MCR mutations can comprise 1, 2, 3, 4, 5, 6, 7, 8, 9, 10, 15, 20, 25, 30, 35, 40, 45, 50, 75, 100 kb or more of genetic material.

In some embodiments, a germline-specific source of nuclease (e.g., Cas9) is preferred. In these embodiments, for example, a cis-regulatory element that is germline specific can be used. In some embodiments, the cis-regulatory element can be testis-specific. In one non-limiting example a testis-specific cis-element in Drosophila and mosquitoes is the beta2-tubulin promoter (i.e., which is expressed selectively in the male germline).

FIGS. 1A-1G are an illustration that depicts one non-limiting example of a MCR. A plasmid or virally-encoded cassette is administered. The MCR cassette can comprise one or more genes, e.g., encoding a Cas9 protein and a guide RNA (gRNA) designed to target a genomic sequence of interest, flanked by homology arms corresponding to the genomic sequences straddling the target site results in cleavage (FIG. 1A). The method uses homology driven insertion (FIGS. 1B, 1C) mechanisms to insert components of the MCR construct, e.g., the sequences encoding the Cas9 and gRNA elements into the targeted locus. The inserted cassette expresses Cas9 protein and gRNA leading to cleavage (FIG. 1D) and homology directed insertion of the cassette into the second allele to render the mutation homozygous (FIGS. 1E, 1F). The MCR construct can further comprise an effector cassette (e.g., a protein or RNA coding sequence) (FIG. 1G).

In certain embodiments, an MCR can also carry additional guide nucleic acids (e.g., guide RNAs) targeting other chromosomal loci for mutagenesis (e.g., via NHEJ) or for propagating a desired chromosomal gene edit with the MCR. In one non-limiting example, one can use one or more gRNA(s) to generate a set of gene edits in one strain of organisms using conventional CRISPR then incorporate the same gRNA(s) into the MCR. One can then cross the MCR strain to the gene edited strain which results in the expression of the MCR derived gRNAs that cut the unedited but not the edited alleles. In subsequent generations, the gRNAs carried by the MCR cut the unedited alleles from the wild-type population and HDR efficiently repairs the lesions using the edited locus as a homology template. Such edits then hijack with the MCR leading to their linked spread in the population (FIG. 9).

In some embodiments, in addition to efficiently copying themselves to a sister chromosome in the germline, MCR elements (see top left panel of FIG. 9) can also be used to drive the spread of unlinked auxiliary elements. In one non-limiting example, FIG. 9 depicts a CHACR element (top middle panel) consisting of three gRNAs. The CHACR element is inserted into the cut site of one of these gRNAs (gRNA2), which is in a different location in the genome than the MCR (which is inserted at a site defined by gRNA1). Thus, like an ERACR, in the presence of an MCR carrying a Cas9 source, the CHACR cuts the opposing chromosome (via cleavage induced by gRNA2) and inserts itself into the resulting DNA gap. In addition, the depicted CHACR carries gRNA3 and gRNA4 which cut at adjacent sites flanking a third edited genomic locus (or existing natural allelic variant—top right panel of FIG. 9). The resulting small deletion (region between the gRNA3 and gRNA4 cut sites) will then be repaired via HDR using the edited chasing mutation sequence. The lower panel of FIG. 9 shows a magnified view of the top right panel indicating gene edited residues as asterisks and the two cleavage sites for gRNA 3 (left) and gRNA4 (right) relative to the sequences of perfect homology mediating HDR repair.

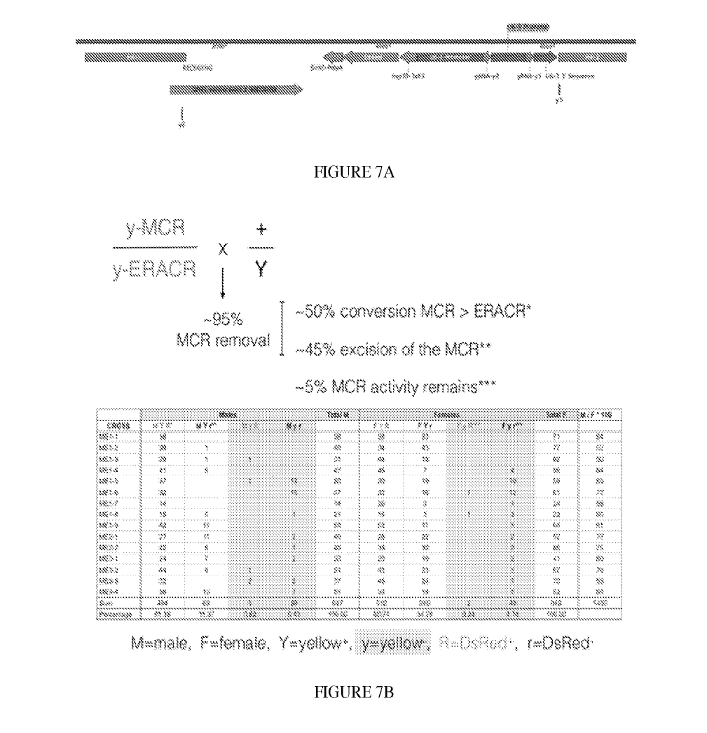

Provided in certain embodiments is also a method for selectively neutralizing or removing the spread of the MCR elements from organisms carrying them. In some cases, the method does not affect organisms lacking MCR elements. This method for selective deletion or neutralization of MCR elements is termed a Neutralizing Chain Reaction (“NCR”) or an Element for Reversing the Autocatalytic Chain Reaction (“ERACR”); the terms are used interchangeably herein. An NCR construct may comprise: 1) one or more guide polynucleotides (e.g., guide RNAs) directing cleavage at the same locus as the MCR element but outside of the MCR element (e.g., to target deletion of MCR sequences from the genome), and 2) homology arms flanking the NCR cassette that directly abut the endonuclease (e.g., Cas9) cut sites determined by the guide polynucleotides (e.g., guide RNAs). An NCR construct may optionally further comprise a recoded gene or cis-regulatory element that restores a genetic function mutated by the MCR of the locus mutated by the MCR element that cannot be cut by the guide polynucleotide(s) (e.g., guide RNA(s)) carried by the MCR element. For example, in the case of an MCR disrupting the coding region of a gene, sequences encoding this gene directly abut the left homology arm (e.g., based on an orientation in which transcription of the gene locus is from left to right) so that it is in-frame with the undisturbed portion of the gene and carries 3′ UTR sequences necessary for producing a functional and stable coding mRNA product (FIG. 7). An NCR construct can optionally comprise an effector cassette. In some cases, an NCR construct does not comprise a gene encoding an endonuclease, such as Cas9. In some cases, an NCR construct comprises a gene encoding an endonuclease, such as Cas9 (e.g., possibly an alternative form of Cas9, a nickase, or a catalytically inert form of Cas9).

An NCR construct can be transfected as a DNA plasmid together with a plasmid source of Cas9 protein into the germline of an organism to obtain transgenic organisms carrying this insertion. Organisms carrying this construct can be crossed with MCR individuals (e.g., released into an environment containing MCR individuals) whereupon the NCR acts on the MCR chromosome to delete the MCR element and restore function of the host locus via the recoded transgene.

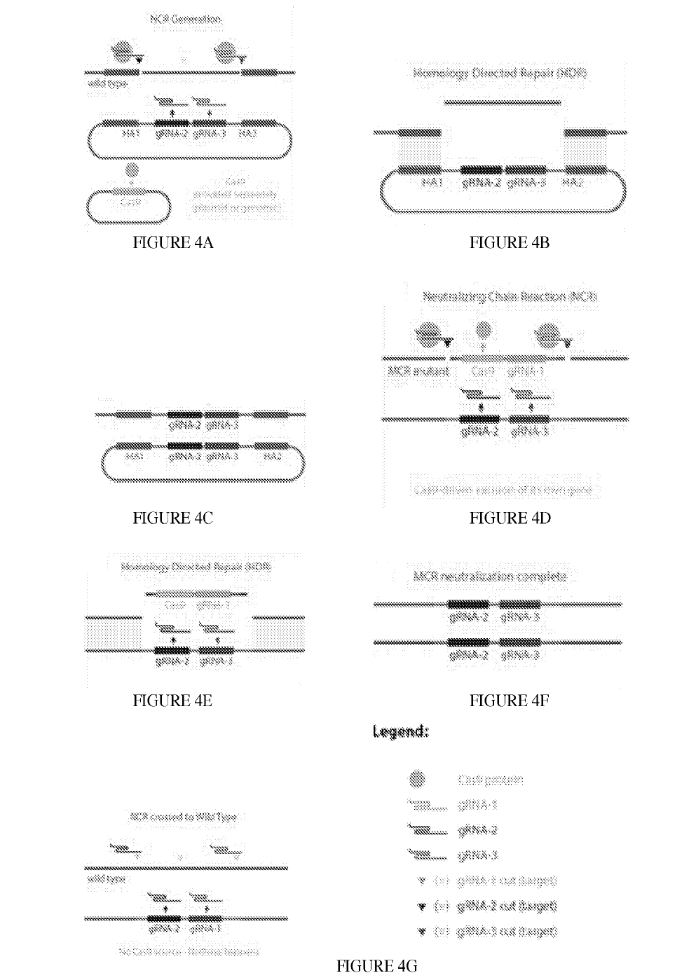

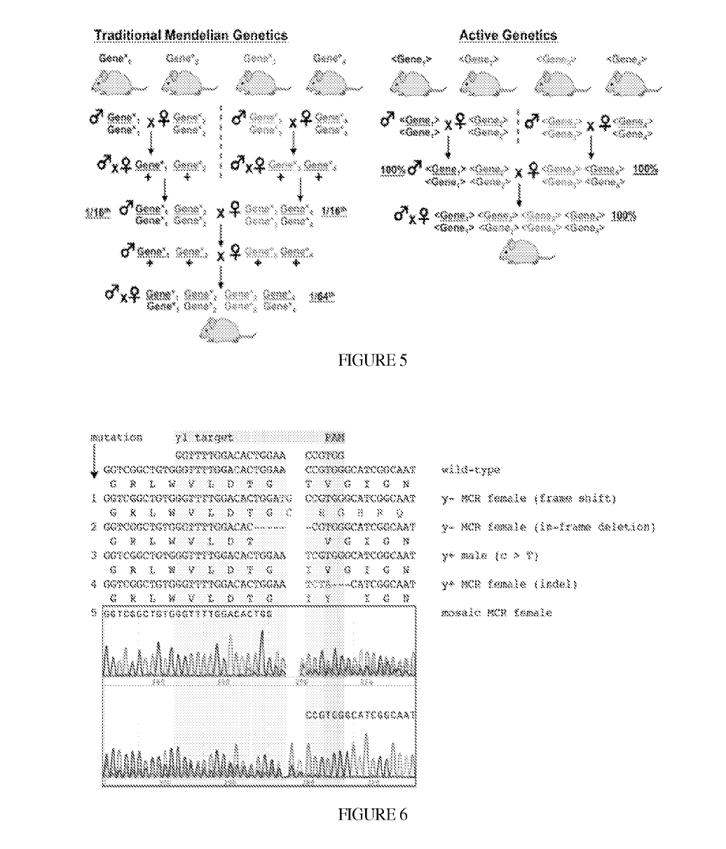

An NCR can delete or neutralize a consequence of having performed MCR (FIG. 4). In some embodiments, the NCR construct is specific for deletion of MCR sequences since it carries guide polynucleotides (e.g., guide RNAs) that lead to cleavage of host sequences flanking the MCR (e.g., thereby cutting out completely) but does not carry the gene encoding Cas9. In some embodiments, the NCR element lacks Cas9 function, so it can only act via its guide polynucleotides (e.g., guide RNAs) in organisms carrying a source of Cas9 (e.g., MCR organisms). In such embodiments, the NCR can cut a distance from the MCR to prevent potential insertion-deletion (indel) type mutations generated by NHEJ (which could destroy the cut sites for the NCR gRNAs) from propagating along with the MCR in subsequent generations. By selecting NCR encoded gRNAs with cut sites far enough away from the MCR insertion one can avoid propagation of NCR-resistant alleles along with the MCR via HDR-mediated resection of the flanking genome sequence since that process typically only extends about 500 bp away from the double stranded DNA break. Therefore, in some embodiments, the NCR may be designed to cut more than: 500 bp, 600 bp, 700 bp, 800 bp, 900 bp, 1000 bp or 2000 bp or more from the flanking genome sequence. In other embodiments, one or more gRNAs carried on the NCR can cut within Cas9 leading to elimination of Cas9 function by causing a non-conservative mutation such as a frameshift mutation or deletion of a portions of the protein coding domain (e.g., two gRNAs designed to cut within Cas9 causing a mutation in one or more of the enzymatically active centers responsible for single stranded cleavage of DNA) or by simply mutating Cas9 via NHEJ (e.g., a single gRNA targeted to nucleotide encoding amino acid residues in one of the two active catalytic centers). In some embodiments, NCRs will be designed to avoid containing sequences homologous to those also present in the MCR (e.g., to avoid homology-dependent cross-over between chromosomes in those regions). FIGS. 7A-7B demonstrate an outcome that can be observed when such sequences are present. In addition, the NCR element can carry a correcting cassette (e.g., coding region of gene or cis-regulatory element) that has been recoded at the original guide-RNA cleavage site(s) to be immune or resistant to MCR cleavage (FIG. 7A). In one non-limiting example, a first generation y-NCR (or y-ERACR) inactivated a corresponding y-MCR with 95% efficiency and that the ERACR could insert in its place, thereby restoring yellow gene function (FIG. 7B).

In one non-limiting example, FIGS. 7A-7B show a first generation ERACR can inactivate the y-MCR with 95% efficiency. FIG. 7A depicts a structure of the y-ERACR. The y1-ERACR consists of a 5′ homology arm covering a portion of the yellow coding region and flanking the gRNA-y2 cut site, a recoded version of the yellow gene that cannot be cleaved by the y1-gRNA carried on the y1-MCR, a DsRed marker gene, two gRNAs directing cleavage on either side of the y-MCR, and a 3′ homology arm flanking the gRNA-2 cut site. This ERACR, when crossed to the MCR can generate Cas9/y2-gRNA and Cas9/y3-gRNA complexes wherein the gRNAs are produced from the ERACR allele and the Cas9 is produced in-trans from the MCR allele. These complexes can then cleave off both sides of the MCR leading to HDR mediated copying of the ERACR into the resulting DNA gap. The recoded yellow sequences carried on the ERACR fused to the adjacent genomic sequences next to the MCR should result in an in-frame active yellow locus thus restoring yellow+activity. FIG. 7B depicts a scheme and data showing that the y-ERACR inactivates the MCR 95% of the time. This result demonstrates that ERACR-derived gRNAs can form active nuclease complexes with MCR-derived Cas9 provided in trans to inactivate the MCR. The fraction of DsRed-, yellow+ male progeny represents the fraction of MCRs inactivated, but not fully converted to ERACR (MYr**) while the fraction of DsRed+, yellow+ male progeny represents the proportion of progeny carrying an intact ERACR (MYR*). The fraction of yellow− female progeny represents the fraction of remaining intact MCR elements (˜5%, FyR and Fyr, ***). These results indicate that while the MCR is reduced to only 5% (95% efficiency in MCR inactivation), only about half the time does this result in clean copying of the ERACR in its place. The most likely basis for the incomplete copying of the ERACR is that this first generation version carries sequences (yellow and U6 pol-III promoters) homologous to those inside the MCR that would permit undesired recombination events within the ERACR. In another non-limiting example, a second generation ERACR, constructed and inserted into the fly genome, lacks these homology sequences (i.e., it carries a fully recoded locus using codons with alternative third position nucleotides and U6 promoters from other fly species with low homology to Drosophila. melanogaster). One can test the efficiency of this ERACR in replacing the MCR. The efficiency will be close to 100%.

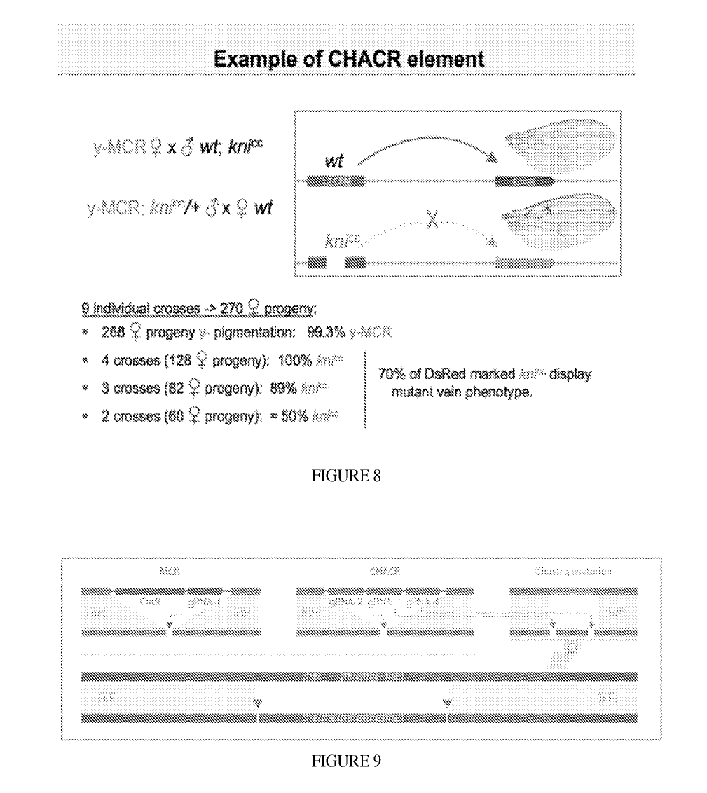

In another non-limiting example, a double-cutting vector was used to carry two gRNAs targeted to a different genomic location than the MCR (referred to as Construct hijacking the Autocatalytic Chain Reaction or CHACR) that can be transmitted with high efficiency to the progeny also lend strong support to the double cut strategy as an efficient means for copying a gRNA-only autocatalytic element from one chromosome to the sister chromosome (FIG. 8). These two properties may selectively correct and neutralize the effects of an MCR element.

In one non-limiting example a CHACR element can be efficiently propagated. In this example, a CHACR (knicc) that carries two gRNAs and a DsRed eye marker was designed to cut at two adjacent sites in a cis-regulatory region (CRM) of the knirps (kni) locus. The CHACR was then inserted into that double-cut. In the presence of an MCR (or any other unlinked source of Cas9) the CHACR then cuts the opposing allele, generates a deletion between the two cut sites and copies itself into the result DNA gap. This genetic event results in loss of CRM activity and corresponding failure to generate a morphological structure dependent on kni function, namely deletion of the second wing vein (*). y-MCR females were crossed to males carrying the knicc CHACR and y-MCR; males were recovered and crossed to wild-type (wt) females. In four such crosses 100% of the progeny inherited the CHACR (DsRed+) and in another 3 crosses 89% progeny inherited the CHACR. In two other examples the CHACR was inherited in a Mendelian fashion, presumably due to the somatic effect of Cas9 in the embryo, which can generate cleavage resistant indels via NHEJ. Use of a strictly germline form of Cas9 will avoid this latter class of crosses, but for most applications the overall excellent efficiency observed in this experiment should be more than adequate to obtain progeny with the desired genotype. These results indicate that double-cut CHACR elements, which function like ERACRs except that they cut at different chromosomal sites than the MCR, can efficiently convert the opposing allele. These findings reinforce our preliminary observations with the first generation ERACR (FIGS. 7A-7B) by supporting the hypothesis that double cut vectors carrying two gRNAs can act in a highly efficient manner to convert the opposing allele.

FIGS. 4A-4G show a scheme outlining the NCR (a.k.a., ERACR). A plasmid (or virally-encoded) cassette carrying two genes encoding two separate gRNA targeting sites flanking the genomic sequence with the previous MCR insertion, flanked by homology arms corresponding to the genomic sequences adjacent to the target sites and identically matching the generated chromosome ends (FIG. 4A) and homology driven insertion (FIG. 4B, 4C) of the core NCR cassette into the targeted wild type locus driven by externally supplied endonuclease (e.g., Cas9) (e.g., by genomic, plasmid, viral, or protein sources). An NCR inserted cassette (FIG. 4C) would be activated starting a NCR only when such animals are crossed with ones carrying its MCR correspondent mutation (FIG. 4D) in which case the reaction would analogously progress to convert the MCR into an NCR allele (FIGS. 4E, 4F) resulting in removal of the endonuclease (e.g., Cas9) gene (FIG. 4F), and thus complete MCR inactivation. In contrast to the MCR, when the NCR is combined with a wild type allele, genome editing does not occur since no source of endonuclease (e.g., Cas9 protein) is available to induce the necessary cleavage (FIG. 4G).

DNA cuts generated by an endonuclease such as Cas9 can be corrected using different cellular repair mechanisms, including: error-prone Non-homologous End Joining (“NHEJ”) and/or Homology Directed Repair (“HDR”). In some embodiments, an MCR or NCR element is integrated into a genome using HDR.

In general, traditional CRISPR applications use NHEJ (which has about 5-20% efficiency). The mutagenic chain reaction or neutralizing chain reaction can use HDR (which has about 90-100% efficiency). The broader term active genetics applies to the use of any construct in which a Cas9 source drives the insertion of a DNA cassette into a particular locus using a gRNA encoded within that cassette. MCR elements, NCR elements, and CopyCat elements are examples of active genetic elements. Active genetic-based applications are more efficient than traditional CRISPR in generating precise genome edits. In some embodiments, the efficiency of an MCR or NCR element integrating into a genome is about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, the efficiency of an MCR or NCR element integrating into a genome is at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, or 99.9%. In some embodiments, the efficiency of an MCR or NCR element integrating into a genome is up to about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%. In some embodiments, the efficiency of allelic conversion of an MCR or NCR element into a genome is about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, the efficiency of allelic conversion of an MCR or NCR element into a genome is at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, or 99.9%. In some embodiments, the efficiency of allelic conversion of an MCR or NCR element into a genome is up to about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%.

MCR may be used to copy DNA fragments of varying size. In some embodiments MCR may be used to copy large DNA fragments, for example, DNA fragments of about 10 kb in length, or DNA fragments of about 17 kb in length. The MCR allows for flexibility in size of DNA of such when engineering applications from environmental pathogens, to plants, to human therapies. In some embodiments, the MCR or NCR element integrated into a genome is about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, or more than 50 kilobases (kb) in length. In some embodiments, the MCR or NCR element integrated into a genome is at least about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, or more than 50 kilobases (kb) in length. In some embodiments, the MCR or NCR element integrated into a genome is up to about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, or more than 50 kilobases (kb) in length. In one non-limiting example, recent experiments have shown that a ˜17 kb MCR propagates via the germline in male and female mosquitoes (Anopheles stephensi) with 99.5% transmission efficiency. In addition, this MCR carries an effector gene cassette previously shown to block the propagation of the malarial parasite Plasmodium falciparum. This gene cassette is inducible by a female mosquito feeding on a blood meal and this induction is also observed for the gene cassette carried by the MCR. See Gantz V, Jasinskiene N, Tatarenkova O, Fazekas A, Macias V M, Bier E, James A A. Highly efficient Cas9-mediated gene drive for population modification of the malaria vector mosquito, Anohpeles stepensi. Proc Natl Acad Sci 2015; In Press, incorporated herein by reference.

MCR elements may nearly double their frequency in a population at each generation, as they may convert chromosomes derived from non-MCR parents to the MCR condition. This results in potent gene drive systems for spreading beneficial genes or exogenous DNA fragments through a population of an organism (e.g., insects that can be as vectors for human disease or insects that are agricultural pests). The same autocatalytic property can be engineered to spread effector transgenes among specific cell populations within an individual (e.g., cancerous cells). This property enables new gene therapy approaches. In some embodiments, the frequency of an MCR or NCR element increases in a population in a generation by a factor of about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more than 3. In some embodiments, the frequency of an MCR or NCR element increases in a population in a generation by a factor of at least about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more than 3. In some embodiments, the frequency of an MCR or NCR element increases in a population in a generation by a factor of up to about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more than 3.

Nucleases are enzymes that cleave the phosphodiester bond within a polynucleotide chain. Endonucleases include, but are not limited to, Cas proteins, restriction endonucleases, meganucleases, homing endonucleases, TAL effector nucleases, and Zinc finger nucleases. Endonucleases include, but are not limited to, Type I, Type II, Type III, Type IV, and Type V endonucleases, any one of which may further include subtypes. Cas proteins include, but are not limited to, Cas1, Cas1B, Cas2, Cas3, Cas3′ (Cas3-prime), Cas3″ (Cas3-double prime), Cas4, Cas5, Cas6, Cas6e (formerly referred to as CasE, Cse3), Cas6f (i.e., Csy4), Cas7, Cas8, Cas8a1, Cas8a2, Cas8b, Cas8c, Cas9 (also known as Csn1 and Csx12), Cas10, Cas10d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, and modified versions thereof. One skilled in the art could choose a nuclease based on various factors, including size, stability, ability to bind to a guide nucleic acid, ability to recognize a target sequence, etc. In some embodiments, the nuclease may be further optimized (e.g., to have a longer half-life, to be codon-optimized for the organism, to further comprise a nuclear localization signal, etc.). In some embodiments, the nuclease can be fused to other functional groups, for example a GFP domain, to visualize the protein.

In some embodiments, the nuclease may be Cas9. In some embodiments, the nuclease may be a Cas9 cloned or derived from a bacteria (S. pyogenes, S. pneumoniae, S. aureus, or S. thermophilus). One skilled in the art will recognize there are many Cas9 nucleases derived from bacteria. One skilled in the art could choose a Cas9 nuclease based on various factors, including size, stability, ability to bind to a guide nucleic acid, ability to recognize a protospacer adjacent motif (i.e., PAM) etc. In some embodiments, the Cas9 nuclease may be further optimized (e.g., to have a longer half-life, to be codon-optimized for the organism, to further comprise a nuclear localization signal, etc.). In some embodiments, the Cas9 nuclease can be fused to other functional groups, for example a GFP domain, to visualize the protein.

A Cas9 protein may recognize a protospacer adjacent motif (PAM) sequence comprising NGG. A Cas9 protein may recognize a protospacer adjacent motif (PAM) sequence that does not comprise NGG. A Cas9 protein may recognize a protospacer adjacent motif (PAM) sequence comprising NNGRRT, such as TTGAAT or TTGGGT.

An endonuclease may have DNA cleavage activity, such as Cas9. In some embodiments, an endonuclease directs cleavage of one or both strands at the location of a target sequence, such as within the target sequence and/or within the complement of the target sequence. In some embodiments, an endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, an endonuclease is mutated with respect to a corresponding wild-type enzyme such that the mutated endonuclease lacks the ability to cleave one or both strands of a target polynucleotide containing a target sequence. For example, an aspartate-to-alanine substitution (e.g., D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, the Cas protein (e.g., Cas9 protein) may be a nickase. In aspects of the invention, nickases may be used for genome editing via homologous recombination. In some embodiments, a Cas9 nickase may be used in combination with guide polynucleotide(s), e.g., two guide polynucleotides, which target respectively sense and antisense strands of the DNA target. Two or more catalytic domains of Cas9 (RuvC I, RuvC II, and RuvC III) may be mutated to produce a mutated Cas9 substantially lacking DNA cleavage activity. In some embodiments, a D10A mutation is combined with one or more of H840A, N854A, or N863A mutations to produce a Cas9 enzyme substantially lacking DNA cleavage activity. In some embodiments, an endonuclease is considered to substantially lacking DNA cleavage activity when the DNA cleavage activity of the mutated endonuclease is less than about 25%, 20%, 15%, 10%, 5%, 4%, 3%, 2%, 1%, 0.5%, 0.1%, 0.05%, 0.01%, or lower than 0.01% with respect to its non-mutated form.

In some embodiments, a gene encoding an endonuclease (e.g., a Cas protein such as Cas9) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cell of interest by replacing at least one codon (e.g., about or more than about 1, 2, 3, 4, 5, 10, 15, 20, 25, 50, or more than 50 codons) of the native sequence with codons that are more frequently or most frequently used in the genes of that host cell while maintaining the native amino acid sequence. Various species may exhibit particular bias for certain codons of a particular amino acid. Codon bias (differences in codon usage between organisms) may correlate with the efficiency of translation of messenger RNA (mRNA), which may depend on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g., 1, 2, 3, 4, 5, 10, 15, 20, 25, 50, more than 50, or all codons) in a sequence encoding an endonuclease correspond to the most frequently used codon for a particular amino acid. In certain embodiments, a gene encoding an endonuclease may not be codon optimized.

In some embodiments, an endonuclease is part of a fusion protein comprising one or more heterologous peptide or protein domains (e.g., about or at least about 1, 2, 3, 4, 5, 6, 7, 8, 9, 10, or more than 10 domains in addition to an endonuclease). An endonuclease fusion protein may comprise any additional peptide or protein sequence, and optionally a linker sequence between any two domains. Examples of peptide or protein domains that may be fused to an endonuclease include, without limitation, epitope tags, reporter gene sequences, localization signals, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), other fluorescent proteins, and autofluorescent proteins including blue fluorescent protein (BFP). An endonuclease may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Examples of localization signals include, but are not limited to, nuclear localization signals (e.g., SV40 large T-antigen, acidic M9 domain of hnRNP A1), cytoplasmic localization signals, mitochondrial localization signals, nuclear export signals, chloroplast localization signals, and endoplasmic reticulum retention signals. In some embodiments, a tagged endonuclease is used to identify the location of a target sequence.

As used herein, the term “guide polynucleotide”, refers to a polynucleotide sequence that can form a complex with an endonuclease (e.g., Cas protein such as Cas9) and enables the endonuclease to recognize and optionally cleave a target site on a polynucleotide such as DNA. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond, or linkage modification such as, but not limited, to locked nucleic acid (LNA), peptide nucleic acid (PNA), bridged nucleic acid (BNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. In some embodiments, the guide polynucleotide does not solely comprise ribonucleic acids (RNAs). In other embodiments, the guide polynucleotide does solely comprise ribonucleic acids (RNAs). A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease. The CER domain of the double molecule guide polynucleotide comprises two separate molecules that are hybridized along a region of complementarity. The two separate molecules can be RNA, DNA, and/or RNA-DNA combination sequences. In some embodiments, the duplex guide polynucleotide does not solely comprise ribonucleic acids (RNAs). In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). In some embodiments, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides).

The guide polynucleotide can also be a single molecule comprising a first nucleotide sequence domain (referred to as Variable Targeting domain or VT domain) that is complementary to a nucleotide sequence in a target DNA and a second nucleotide domain (referred to as endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides).

The term “variable targeting domain” or “VT domain” is used interchangeably herein and refers to a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

In some embodiments, an MCR or NCR construct or element comprises about 1, 2, 3, 4, 5, 6, 7, 8, 9, 10, 15, 20, 25, 30, 35, 40, 45, 50, or more than 50 guide polynucleotides. In some embodiments, an MCR or NCR construct or element comprises at least about 1, 2, 3, 4, 5, 6, 7, 8, 9, 10, 15, 20, 25, 30, 35, 40, 45, 50, or more than 50 guide polynucleotides. In some embodiments, an MCR or NCR construct or element comprises up to about 1, 2, 3, 4, 5, 6, 7, 8, 9, 10, 15, 20, 25, 30, 35, 40, 45, 50, or more than 50 guide polynucleotides.

In general, a guide polynucleotide is any polynucleotide sequence having sufficient complementarity with a target polynucleotide sequence to hybridize with the target sequence and direct sequence-specific binding of a CRISPR complex to the target sequence. In some embodiments, the degree of complementarity between a guide polynucleotide and its corresponding target sequence, when optimally aligned using a suitable alignment algorithm, is about or more than about 50%, 55%, 60%, 65%, 70%, 75%, 80%, 85%, 90%, 95%, 96%, 97%, 98%, 99%, 99.5%, 99.9%, or 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide polynucleotide is about or at least about 5, 10, 11, 12, 13, 14, 15, 16, 17, 18, 19, 20, 21, 22, 23, 24, 25, 26, 27, 28, 29, 30, 35, 40, 45, 50, 75, or more than 75 nucleotides in length. In some embodiments, a guide polynucleotide is up to about 75, 50, 45, 40, 35, 30, 25, 20, 15, 12, or fewer than 12 nucleotides in length. The ability of a guide polynucleotide to direct sequence-specific binding of a CRISPR complex to a target sequence may be assessed by any suitable assay.

A guide polynucleotide may be selected to target any target sequence. In some embodiments, the target sequence is a sequence within a genome of a cell. Exemplary target sequences include those that are unique in the target genome.

A homology arm may be about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, or more than 500 nucleotides in length. In some embodiments, homology arms on an MCR or NCR construct are the same length, similar lengths, or different lengths. In some embodiments, the degree of complementarity between a homology arm and its corresponding target sequence, when optimally aligned using a suitable alignment algorithm, is about or more than about 50%, 55%, 60%, 65%, 70%, 75%, 80%, 85%, 90%, 95%, 96%, 97%, 98%, 99%, 99.5%, 99.9%, or 100%. In some instances, the homology arms directly abut the endonuclease cleavage sites. In some embodiments of any one of the methods or constructs described herein, the homology arms directly abut the endonuclease cut site(s) determined by the at least one guide polynucleotide, or are separated by less than 100, 75, 50, 40, 30, 20, 10, 9, 8, 7, 6, 5, 4, 3, 2, or 1 nucleic acids.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

Methods of introducing a nucleic acid into a host cell are known in the art, and any known method can be used to introduce a nucleic acid (e.g., an expression construct) into a cell. Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

FIGS. 2A-2I show an experimental demonstration of MCR in Drosophila. FIG. 2A) Standard Mendelian inheritance of a homozygous trait in which all offspring are heterozygous for that trait. FIG. 2B) MCR based inheritance results in the initially heterozygous allele converting the second allele and the individual becoming homozygous (or nearly so) for that mutation. FIG. 2C) Diagram of y-MCR construct. The two y homology arms flanking the vasa-Cas9 and y− gRNA transgenes are indicated as well as the locations of the PCR primers used for analysis of the genomic insertion site which are listed in the methods section. FIG. 2D) PCR analysis of y-MCR F1 ♀ and a sibling ♂ showing functional bands corresponding to insertion of the y-MCR construct into the chromosomal y locus as well a band amplified from the y locus without an MCR insertion. As expected, y-MCR F1 ♂ with a single X− chromosome display only MCR derived PCR products, while both MCR and non-insertional alleles were amplified from all 6 tested y-MCR F1 ♀. FIG. 2E) A low magnification view of flies emerging from the cross of y−w−MCR F1 ♀ to y+w− ♂ showing that almost all progeny have the y− phenotype. FIG. 2F) A high magnification view of a full body y−w−MCR F1 ♀. FIG. 2G) A rare mosaic female with 50% of the body y− and 50% y+ with the dividing line running the length of the body. FIG. 2H) A y+w− control fly. FIG. 2I) Example of DNA sequences at junction of homology arms with an MCR element (y-MCR) illustrating how the homology arms precisely abut the gRNA cut site to the nucleotide.

MCR constructs may be used to disperse (or drive) transgenes into animal or plant pest populations to combat propagation of insect borne pathogens or diseases (e.g., Malaria), to selectively inhibit propagation of insect pests in crop fields, or to help control weeds (FIGS. 3A, 3B). An MCR construct supplied to somatic cells within an individual via a replicating vector (e.g., a virus) could insert into diseased cells carrying specific sequences (e.g., retroviral insertions or cancer cell specific mutations) and then spread to other cells within that organism (FIGS. 3C, 3D). Such constructs by virtue of carrying effector cassettes could then be engineered to combat the disease by killing the diseased cells (e.g., by inducing production of a toxin or a cell surface molecule to alert the host immune system) or by altering them in some other way (e.g., by repairing a gene or restoring a necessary cellular function). In addition, MCR elements may be used for gene therapy purposes to either fix mutant genes or eliminate gene functions contributing to a disease state.

FIGS. 3A-3D describe some potential applications of MCR. FIG. 3A) Application of MCR to attenuate mosquito borne malaria in which an effector cassette encoding the SM1 peptide, which is conditionally activated by a blood meal (AgCP promoter) or a single chain antibody (scFvs) directed against the malarial agent P. falciparum (7), is inserted along with core MCR components (Cas9 and gRNA) into a non-coding region of the mosquito genome. The SM1 peptide limits passage of P. falciparum through the gut, a required step in its exploitation of that vector host (6). Spread of such an MCR construct through the mosquito population should follow an exponential trajectory that could lead to complete spread throughout a host population in 35 generations if transmission is as efficient as shown for the y-MCR element in Drosophila, and making the assumption of no reduced fitness being associated with the MCR, a single individual carrying an MCR construct could spread the MCR element to an entire population. It is notable that in such models, the percentage of the MCR element in the population could increase from 1% to 100% in only 9 generations. FIG. 3B) A scheme similar to that in panel A wherein transgenic crops produce a signal (e.g., hormone) that activates expression of toxin to control a specific pest engineered to spread an MCR cassette carrying the toxin. FIG. 3C) MCR based spread of an Integrase-deficient Cas9/gRNA-dependent retroviral (e.g., HIV) construct directing its insertion into a chromosomal inserted provirus thereby rendering that proviral element inactive. Induction and maturation of such targeted proviruses should lead to the production of assembled viruses which could then infect all other CD4+ helper T-cells but only integrates into the genomes of cells carrying proviral insertions. This within-organism spread of the MCR construct could eventually incapacitate all proviruses leading to the eventual clearance of the HIV infection. FIG. 3D) An analogous retro-virally propagated MCR element directs its insertion into a cancer-specific genomic sequence. Infection and spread of this element throughout the body should lead to its selective insertion in cancer cells (in primary and metastatic tumors). When testing of patient cells indicates that the MCR has spread effectively to all cancer cells, an effector cassette carried by the MCR could be activated (e.g., by a hormone) to induce apoptosis or flag cells for destruction by the immune system.

For example, if a gene is introduced, which when expressed prevents mosquitoes from harboring the malaria parasite, a few individuals carrying such an expression cassette on such a gene-drive construct can be released to spread that cassette into that population exponentially. The mutant offspring can then replace the wild-type species in about 10-20 generations.

Other applications involve getting the mutation to spread within cells of a single individual afflicted with a disease such as HIV or cancer. The invention targets insertion of the construct into DNA sequences that are specific to diseased cells and then carry some type of cassette that could kill, fix, or reprogram the diseased cells.

Targeting the HIV reservoir: Retroviruses such as HIV insert into the host genome. As shown in FIG. 3C, an MCR element may direct its insertion into the HIV integrase gene and replace the gene's function with CRISPR/Cas9-mediated insertion. If a construct of this kind is designed such that the Cas9 and gRNAs are packaged within HIV viral particles, then the virus will be able to infect all CD4+ cells, but only integrate into those carrying a HIV provirus in the genome. Virus produced by such targeted MCR elements can then replicate and spread to other helper T-cells but only integrate into those with a proviral insertion. This process will continue until cells carrying the provirus in their genome are neutralized. Even though HIV reservoir cells may be quiescent and HDR-mediated allelic conversion may require DNA replication, methods are available for inducing reservoir cells to re-enter the cell cycle, which then may allow the chain of events described above to proceed. Similar strategies target other retroviruses or DNA viruses that accumulate multiple copies of their genomes within cells (e.g., Herpes viruses).

Selectively Targeting Cancer Cells:

MCRs designed to spread between cells in the body can be developed that target nucleotide differences between the cancer cell and normal cells, which can now be rapidly detected by deep sequencing. Types of cancer in which cancer-cell specific sequences can be identified (e.g., chromosomal rearrangements) can be targeted by a construct comprising a cancer-specific gRNA carried by an MCR packaged in an Integrase-deficient retrovirus or adenovirus. Such an MCR-viral construct can infect both normal and cancer cells in the patient, but only insert into the genome of cancer cells (FIG. 3D). For an element engineered to replicate and spread from cell-to-cell, an initial infection of only a small subset of cancer cells may result in spread of the MCR-virus until the great majority of cancer cells contained the construct even if the primary tumor had metastasized. Infection of cancer cells can be readily monitored by physicians and once MCR-viral delivery became widespread, the cancer would be progressively attacked by activating drug-inducible effectors carried by the MCR. Such effectors can include toxins, agents triggering apoptosis, or cellular antigens that flag cells for immune recognition. Similar generalized strategies to combat cancer that are independent of the type of cancer or stage of cancer progression may be targeted using MCRs.

Cancers include, but are not limited to, Acute lymphoblastic leukemia (ALL); Acute myeloid leukemia; Adrenocortical carcinoma; AIDS-related cancers; AIDS-related lymphoma; Anal cancer; Appendix cancer; Astrocytoma, childhood cerebellar or cerebral; Basal-cell carcinoma; Bile duct cancer, extrahepatic; Bladder cancer; Bone tumor, osteosarcoma/malignant fibrous histiocytoma; Brain cancer; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Brainstem glioma; Breast cancer; Bronchial adenomas/carcinoids; Burkitt's lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/malignant glioma, childhood; Cervical cancer; Childhood cancers; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, childhood; Extragonadal germ cell tumor; Extrahepatic bile duct cancer; Eye cancer, intraocular melanoma; Eye cancer, retinoblastoma; Gallbladder cancer; Gastric (stomach) cancer; Gastric carcinoid; Gastrointestinal carcinoid tumor; Gastrointestinal stromal tumor (GIST); Germ cell tumor: extracranial, extragonadal, or ovarian; Gestational trophoblastic tumor; Glioma of the brain stem; Glioma, childhood cerebral astrocytoma; Glioma, childhood visual pathway and hypothalamic; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular melanoma; Islet cell carcinoma (endocrine pancreas); Kaposi sarcoma; Kidney cancer (renal cell cancer); Laryngeal cancer; Leukaemia, acute lymphoblastic (also called acute lymphocytic leukaemia); Leukaemia, acute myeloid (also called acute myelogenous leukemia); Leukaemia, chronic lymphocytic (also called chronic lymphocytic leukemia); Leukaemias; Leukemia, chronic myelogenous (also called chronic myeloid leukemia); Leukemia, hairy cell; Lip and oral cavity cancer; Liposarcoma; Liver cancer (primary); Lung cancer, non-small cell; Lung cancer, small cell; Lymphoma, AIDS-related; Lymphoma, Burkitt; Lymphoma, cutaneous T-Cell; Lymphoma, Hodgkin; Lymphoma, primary central nervous system; Lymphomas; Lymphomas, Non-Hodgkin (an old classification of all lymphomas except Hodgkin's); Macroglobulinemia, Waldenstrom; Male breast cancer; Malignant fibrous histiocytoma of bone/osteosarcoma; Medulloblastoma, childhood; Melanoma; Melanoma, intraocular (eye); Merkel cell cancer; Mesothelioma, adult malignant; Mesothelioma, childhood; Metastatic squamous neck cancer with occult primary; Mouth cancer; Multiple endocrine neoplasia syndrome, childhood; Multiple myeloma/plasma cell neoplasm; Mycosis fungoides; Myelodysplastic syndromes; Myelodysplastic/myeloproliferative diseases; Myelogenous leukemia, chronic; Myeloid leukemia, adult acute; Myeloid leukemia, childhood acute; Myeloma, multiple (cancer of the bone-marrow); Myeloproliferative disorders, chronic; Nasal cavity and paranasal sinus cancer; Nasopharyngeal carcinoma; Neuroblastoma; Non-Hodgkin lymphoma; Non-small cell lung cancer; Oligodendroglioma; Oral cancer; Oropharyngeal cancer; Osteosarcoma/malignant fibrous histiocytoma of bone; Ovarian cancer; Ovarian epithelial cancer (surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (melanoma); Skin cancer (non-melanoma); Skin carcinoma, Merkel cell; Small cell lung cancer; Small intestine cancer; Soft tissue sarcoma; Squamous cell carcinoma; Squamous neck cancer with occult primary, metastatic; Stomach cancer; Supratentorial primitive neuroectodermal tumor, childhood; T-Cell lymphoma, cutaneous; Testicular cancer; Throat cancer; Thymoma and thymic carcinoma; Thymoma, childhood; Thyroid cancer; Thyroid cancer, childhood; Transitional cell cancer of the renal pelvis and ureter; Trophoblastic tumor, gestational; Unknown primary site, cancer of, childhood; Unknown primary site, carcinoma of, adult; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenström macroglobulinemia; Wilms tumor (kidney cancer), childhood; and any combination thereof.

Targeting Microorganisms.

An MCR element may direct its insertion into one or more genes of a microorganism, for example, to treat an disease or illness, decrease pathogenicity, decrease virulence, decrease or reverse resistance to an antimicrobial (e.g., antibacterial, antifungal, antiviral, antiparasitic), decrease colonization, decrease transmission, decrease persistence, decrease replication, and/or kill a microorganism. Some non-limiting examples of a microorganism or microbe include bacteria, archaea, protozoa, protists, fungus, algae, virus, retrovirus, pathogen, or parasite. In some cases, the microorganism or microbe is a prokaryote. In some cases, the microorganism or microbe is a eukaryote. Some non-limiting examples of bacteria include Bacillus, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Staphyloccus Aures, Streptococcus, Treponema, Vibrio, and Yersinia. Some non-limiting examples of fungi include Candida, Aspergillus, Cryptococcus, Histoplasma, Pneumocystis, and Stachybotrys. In some instances, the microbe or microorganism detected by the methods provided herein is a drug-resistant microbe or multi-drug resistant pathogen. Non-limiting examples of drug-resistant or multi-drug resistant pathogens include: In some cases, drug-resistant strains of Clostridium difficile (C. difficile), carbapenem-resistant Enterobacteriaceae (CRE), drug-resistant Neisseria, gonorrhoeae (cephalosporin resistant), multidrug-resistant Acinetobacter, drug-resistant Campylobacter, fluconazole-resistant Candida (a fungus), extended spectrum β-lactamase producing Enterobacteriaceae (ESBLs), vancomycin-resistant Enterococcus (VRE), multidrug-resistant Pseudomonas aeruginosa, drug-resistant non-typhoidal Salmonella, drug-resistant Salmonella Typhi, drug-resistant Shigella, methicillin-resistant Staphylococcus aureus (MRSA), drug-resistant Streptococcus pneumonia, drug-resistant tuberculosis (MDR and XDR), multi-drug resistant Staphylococcus aureus, vancomycin-resistant Staphylococcus aureus (VRSA), erythromycin-resistant Streptococcus Group A, or clindamycin-resistant Streptococcus Group B.

In some cases, a virus is a retrovirus or lentivirus. In some cases, the virus is a member of Group I, Group II, Group III, Group IV, Group V, Group VI, or Group VII in the Baltimore virus classification system. In some cases, a virus is a member of the family Adenoviridae, Anelloviridae, Arenaviridae, Astroviridae, Bunyaviridae, Caliciviridae, Coronaviridae, Filoviridae, Flaviviridae, Hepadnaviridae, Hepeviridae, Herpesviridae, Orthomyxoviridae, Papillomaviridae, Papovaviridae, Paramyxoviridae, Parvoviridae, Picornaviridae, Polyomaviridae, Poxviridae, Reoviridae, Retroviridae, Rhabdoviridae, or Togaviridae. In some cases, a virus is Adenovirus, Amur virus, Andes virus, Animal virus, Astrovirus, Avian nephritis virus, Avian orthoreovirus, Avian Reovirus, Banna virus, Bas-Congo virus, Bat-borne virus, BK virus, Blueberry shock virus, Chicken anaemia virus, Bovine adenovirus, Bovine coronavirus, Bovine herpesvirus 4, Bovine parvovirus, Bulbul coronavirus HKU11, Carrizal virus, Catacamas virus, Chandipura virus, Channel catfish virus, Choclo virus, Coltivirus, Coxsackievirus, Cricket paralysis virus, Crimean-Congo hemorrhagic fever virus, Cytomegalovirus, dengue virus, Dobrava-Belgrade virus, Ebola virus, Ebolavirus, El Moro Canyon virus, Elephant endotheliotropic herpesvirus, Epstein-Barr virus, Feline leukemia virus, Foot-and-mouth disease virus, Gou virus, Guanarito virus, Hantaan River virus, Hantavirus, HCoV-EMC/2012, Hendra virus, Henipavirus, Hepatitis A virus, Hepatitis B virus, Hepatitis C virus, Hepatitis D, Hepatitis E virus, Herpes simplex type 1, Herpes simplex type 2, Herpes simplex virus type 1, Herpes simplex virus type 2, HIV, Human astrovirus, Human bocavirus, Human cytomegalovirus, Human herpesvirus type 8, Human herpesvirus type 8, Human immunodeficiency virus (HIV), Human metapneumovirus, Human papillomavirus, Imjin virus, Influenza virus, Isla Vista virus, JC virus, Junin virus, Khabarovsk virus, Koi herpes virus, Kunjin virus, Lassa virus, Limestone Canyon virus, Lloviu cuevavirus, Lloviu virus, Lujo virus, Machupo virus, Magboi virus, Marburg marburgvirus, Marburg virus, Marburgvirus, Measles virus, Melaka virus, Menangle virus, Middle East respiratory syndrome coronavirus, Miniopterus Bat coronavirus 1, Miniopterus Bat coronavirus HKU8, Monkeypox virus, Monongahela virus, Muju virus, Mumps virus, Nipah virus, Norwalk virus, Orbivirus, Parainfluenza virus, Parvovirus B19, Phytoreovirus, Pipistrellus bat coronavirus HKU5, Poliovirus, Porcine adenovirus, Prospect Hill virus, Qalyub virus, Rabies virus, Ravn virus, Respiratory syncytial virus, Reston virus, Reticuloendotheliosis virus, Rhinolophus Bat coronavirus HKU2, rhinovirus, Roseolovirus, Ross River virus, Rotavirus, Rousettus bat coronavirus HKU9, Rubella virus, Saaremaa virus, Sabin virus, Sangassou virus, Scotophilus Bat coronavirus 512, Serang virus, Severe acute respiratory syndrome virus, Shope papilloma virus, Simian foamy virus, Sin Nombre virus, Smallpox, Soochong virus, Sudan ebolavirus, Sudan virus, Taï Forest ebolavirus, Taï Forest virus, Tanganya virus, Thottapalayam virus, Topografov virus, Tremovirus, Tula virus, Turkey coronavirus, Turkeypox virus, Tylonycteris bat coronavirus HKU4, Varicella zoster virus, Varicella-zoster virus, West Nile virus, Woodchuck hepatitis virus, yellow fever virus, or Zaire ebolavirus.

Some non-limiting examples of a pathogen include a virus, bacterium, prion, fungus, parasite, protozoan, and microbe. Some non-limiting examples of pathogens include Acanthamoeba, Acari, Acinetobacter baumannii, Actinomyces israelii, Actinomyces gerencseriae, Propionibacterium propionicus, Actinomycetoma, Eumycetoma, Adenoviridae, Alphavirus, Anaplasma genus, Anaplasma phagocytophilum, Ancylostoma braziliense, Ancylostoma duodenale, Necator americanus, Angiostrongylus costaricensis, Anisakis, Arachnida Ixodidae, Argasidae, Arcanobacterium haemolyticum, Archiacanthocephala, Moniliformis moniliformis, Arenaviridae, Ascaris lumbricoides, Ascaris sp. Ascaris lumbricoides, Aspergillus genus, Astroviridae, Babesia B. divergens, B. bigemina, B. equi, B. microfti, B. duncani, Babesia genus, Bacillus anthracis, Bacillus cereus, Bacteroides genus, Balamuthia mandrillaris, Balantidium coli, Bartonella henselae, Baylisascaris genus, Baylisascaris procyonis, Bertiella mucronata, Bertiella studeri, BK virus, Blastocystis, Blastocystis hominis, Blastomyces dermatitidis, Bordetella pertussis, Borrelia burgdorferi, Borrelia species, Borrelia genus, Brucella genus, Brugia malayi, Brugia timori, Bunyaviridae, Burkholderia cepacia, Burkholderia species, Burkholderia mallei, Burkholderia pseudomallei, Caliciviridae, Campylobacter genus, Candida albicans, Candida species, Cestoda, Taenia multiceps, Chlamydia trachomatis, Chlamydia trachomatis, Neisseria gonorrhoeae, Chlamydophila pneumoniae, Chlamydophila psittaci, Cimicidae Cimex lectularius, Clonorchis sinensis; Clonorchis viverrini, Clostridium botulinum, Clostridium difficile, Clostridium perfringens, Clostridium perfringens, Clostridium species, Clostridium tetani, Coccidioides immitis, Coccidioides posadasii, Cochliomyia hominivorax, Colorado tick fever virus (CTFV), Coronaviridae, Corynebacterium diphtheriae, Coxiella bumetii, Crimean-Congo hemorrhagic fever virus, Cryptococcus neoformans, Cryptosporidium, Cryptosporidium genus, Cyclospora cayetanensis, Cytomegalovirus, Demodex folliculorum/brevis/canis, Dengue viruses (DEN-1, DEN-2, DEN-3 and DEN-4), Flaviviruses, Dermatobia hominis, Dicrocoelium dendriticum, Dientamoeba fragilis, Dioctophyme renale, Diphyllobothrium, Diphyllobothrium latum, Dracunculus medinensis, Ebolavirus (EBOV), Echinococcus genus, Echinococcus granulosus, Echinococcus multilocularis, E. vogeli, E. oligarthrus, Ehrlichia chaffeensis, Ehrlichia ewingii, Ehrlichia genus, Entamoeba histolytica, Entamoeba histolytica, Enterobius vermicularis, Enterobius gregorii, Enterococcus genus, Enterovirus genus, Enteroviruses, Coxsackie A virus, Enterovirus 71 (EV71), Epidermophyton floccosum, Trichophyton rubrum, Trichophyton mentagrophytes, Epstein-Barr Virus (EBV), Escherichia coli 0157:H7, 0111 and 0104:H4, Fasciola hepatica, Fasciola gigantica, Fasciolopsis buski, Filarioidea superfamily, Filoviridae, Flaviviridae, Fonsecaea pedrosoi, Francisella tularensis, Fusobacterium genus, Geotrichum candidum, Giardia intestinalis, Giardia lamblia, Gnathostoma spinigerum, Gnathostoma hispidum, Group A Streptococcus, Staphylococcus, Guanarito virus, Haemophilus ducreyi, Haemophilus influenzae, Halicephalobus gingivalis, Heartland virus, Helicobacter pylori, Hepadnaviridae, Hepatitis A Virus, Hepatitis B Virus, Hepatitis C Virus, Hepatitis D Virus, Hepatitis E Virus, Hepeviridae, Herpes simplex virus 1 and 2 (HSV-1 and HSV-2), Herpesviridae, Histoplasma capsulatum, HIV (Human immunodeficiency virus), Hortaea werneckii, Human bocavirus (HBoV), Human herpesvirus 6 (HHV-6), Human herpesvirus 7 (HHV-7), Human metapneumovirus (hMPV), Human papillomavirus (HPV), Human parainfluenza viruses (HPIV), Hymenolepis nana, Hymenolepis diminuta, Isospora belli, JC virus, Junin virus, Kingella kingae, Klebsiella granulomatis, Lassa virus, Legionella pneumophila, Leishmania, Leptospira genus, Linguatula serrata, Listeria monocytogenes, Loa loa filaria, Lymphocytic choriomeningitis virus (LCMV), Machupo virus, Malassezia genus, Mansonella streptocerca, Marburg virus, Measles virus, Metagonimus yokagawai, Microsporidia phylum, Middle East respiratory syndrome coronavirus, Molluscum contagiosum virus (MCV), Monkeypox virus, Mucorales order (Mucormycosis), Entomophthorales order (Entomophthoramycosis), Mumps virus, Mycobacterium leprae, Mycobacterium lepromatosis, Mycobacterium tuberculosis, Mycobacterium ulcerans, Mycoplasma pneumoniae, Naegleria fowleri, Neisseria gonorrhoeae, Neisseria meningitidis, Nocardia asteroides, Nocardia species, Oestroidea, Calliphoridae, Sarcophagidae, Onchocerca volvulus, Opisthorchis viverrini, Opisthorchis felineus, Clonorchis sinensis, Orthomyxoviridae, Papillomaviridae, Paracoccidioides brasiliensis, Paragonimus africanus; Paragonimus caliensis; Paragonimus kellicotti; Paragonimus skrjabini; Paragonimus uterobilateralis, Paragonimus westermani, Paragonimus species, Paramyxoviridae, parasitic dipterous fly larvae, Parvoviridae, Parvovirus B19, Pasteurella genus, Pediculus humanus, Pediculus humanus capitis, Pediculus humanus corporis, Phthirus pubis, Picornaviridae, Piedraia hortae, Plasmodium falciparum, Plasmodium vivax, Plasmodium ovale curtisi, Plasmodium ovale wallikeri, Plasmodium malariae, Plasmodium knowlesi, Plasmodium genus, Pneumocystis jirovecii, Poliovirus, Polyomaviridae, Poxviridae, Prevotella genus, PRNP, Pthirus pubis, Pulex irritans, Rabies virus, Reoviridae, Respiratory syncytial virus (RSV), Retroviridae, Rhabdoviridae, Rhinosporidium seeberi, Rhinovirus, rhinoviruses, coronaviruses, Rickettsia akari, Rickettsia genus, Rickettsia prowazekii, Rickettsia rickettsii, Rickettsia typhi, Rift Valley fever virus, Rotavirus, Rubella virus, Sabia, Salmonella enterica subsp. enterica, serovar typhi, Salmonella genus, Sarcocystis bovihominis, S. arcocystis suihominis, S. arcoptes scabiei, SARS coronavirus, Schistosoma genus, Schistosoma haematobium, Schistosoma japonicum, Schistosoma mansoni and Schistosoma intercalatum, Schistosoma mekongi, Schistosoma sp., Shigella genus, Sin Nombre virus, Spirometra erinaceieuropaei, Sporothrix schenckii, Staphylococcus genus, Streptococcus agalactiae, Streptococcus pneumoniae, Streptococcus pyogenes, Strongyloides stercoralis, Taenia genus, Taenia saginata, Taenia solium, the bacterial family Enterobacteriaceae, Thelazia californiensis, Thelazia callipaeda, Togaviridae, Toxocara canis, Toxocara cati, Toxoplasma gondii, Treponema pallidum, Trichinella spiralis, Trichinella britovi, Trichinella nelsoni, Trichinella nativa, Trichobilharzia regenti, Schistosomatidae, Trichomonas vaginalis, Trichophyton genus, Trichophyton rubrum, Trichophyton tonsurans, Trichosporon beigelii, Trichuris trichiura, Trichuris trichiura, Trichuris vulpis, Trypanosoma brucei, Trypanosoma cruzi, Tunga penetrans, Ureaplasma urealyticum, Varicella zoster virus (VZV), Variola major, Variola minor, Venezuelan equine encephalitis virus, Vibrio cholerae, West Nile virus, Wuchereria bancrofti, Wuchereria bancrofti, Brugia malayi, Yellow fever virus, Yersinia enterocolitica, Yersinia pestis, and Yersinia pseudotuberculosis.

Combating Insect Borne Diseases:

MCR elements can be designed that block disease transmission. In the case of malaria, for example, MCR elements may be designed to carry anti-malarial effector cassettes, which encode factors that may prevent the malarial parasite from completing its life cycle, but may not harm the mosquito and hence may have a neutral effect on the environment (FIG. 3A). Mosquitoes carrying such a construct may be released into an area where malaria is endemic. Mosquitoes may then mate with indigenous mosquitoes and spread the MCR construct exponentially through the population in as few as 10 generations (FIG. 2A). This goal may be accomplished in a single season since it is estimated that mosquitoes complete 10-20 reproductive cycles per year. As more mosquitoes in the treated area carry the construct, propagation of malaria should be greatly reduced or eliminated.

Similar strategies could be devised to combat other insect borne diseases. Insects that carry insect borne diseases include, but are not limited to, the mosquito, tick, flea, lice, Culicoid midge, sandfly, Tsetse fly, and bed bug. Insect borne diseases include, but are not limited to, mosquito borne diseases, tick borne diseases, flea borne diseases, lice borne diseases, Culicoid midge borne diseases, sandfly borne diseases, Tsetse fly borne diseases, bed bug borne diseases, and any combination thereof. Examples of insect borne diseases include, but are not limited to, African horse sickness, babesiosis, bluetongue disease, tick-borne encephalitis, Rickettsial diseases (e.g., typhus, rickettsialpox, Boutonneuse fever, African tick bite fever, Rocky Mountain spotted fever), Crimean-Congo hemorrhagic fever, ehrlichiosis, Southern tick-associated rash illness, tick-borne relapsing fever, tularemia, lice infestation, heartland virus, plague, Trypanosomiasis, sleeping sickness, leishmaniasis, Chagas disease, and Lyme disease. Mosquito borne diseases include, but are not limited to, malaria, dengue fever, yellow fever, chikungunya, dog heartworm, Eastern equine encephalitis, epidemic polyarthritis, filariasis, Rift Valley fever, Ross River fever, St. Louis encephalitis, Japanese encephalitis, pogosta disease, LaCrosse encephalitis, Western equine encephalitis, and West Nile virus.

Treating Diseases or Conditions:

MCR elements can be designed that treat diseases or conditions by selectively adding, deleting, or mutating genes. For example, genes that encode immunogenic proteins may be targeted to reduce or eliminate immunogenicity. Allergens in food may be reduced by targeting the genes encoding the allergen in the organism (e.g., peanut, tree nut, cow (or other source of milk), chicken (or other source of egg), wheat, soy, fish, shellfish) from which the food was derived. Specific cells may be targeted, such as beta cells (role in diabetes) or cells and/or genes involved in autoimmune disorders.

Controlling Agriculture Pest Species: Agriculture pests and invasive species cause over $3 billion of damage to crops per year. MCRs and/or NCRs targeting one or more genes, for example those required for female fertility or survival, may reduce the damage caused by many of these pests.

For instance, MCRs can suppress crop pests actively attacking a crop of interest or be used for weed control. This strategy closely parallels that illustrated above for combating malaria. For example, the spotted wing fly (Drosophila suzukii), which is related to the laboratory fruit fly (Drosophila melanogaster), may be targeted. D. suzukii entered the U.S. in 2008 and in 2010 was estimated to cause over $500 million of damage to soft fruits (strawberries, other berries, grapes, cherries) in Pacific coast states, amounting to nearly 20% of this $2.5 billion industry. The genome sequence of D. suzukii has been determined, and MCR constructs can be generated to test for control and eradication of this invasive pest. Other pests that may also be targeted include, but are not limited to, the Medfly (≈$1.2 billion damage/yr), olive fly (can reduce oil production by as much as 80%), pea leaf miner (a fly causing over $1.5 billion of crop damage), and Asian tiger mosquito (a vector for encephalitis, dengue fever, yellow fever and dog heartworm). Pests or weeds that are resistant to pesticides or herbicides (e.g., glyphosate), respectively, may also be targeted by MCRs and/or NCRs. For example, MCRs may replace resistant alleles to restore susceptibility to a pesticide or herbicide. Resistant pests that may be targeted include, but are not limited to, the western corn rootworm, horseweed, pigweed, Amaranthus hybridus (syn: quitensis) (Smooth Pigweed); Amaranthus palmeri (Palmer Amaranth); Amaranthus spinosus (Spiny Amaranth); Amaranthus tuberculatus (=A. rudis) (Tall Waterhemp); Ambrosia artemisiifolia (Common Ragweed); Ambrosia trifida (Giant Ragweed); Bidens pilosa (Hairy Beggarticks); Brachiaria eruciformis (Sweet Summer Grass); Bromus diandrus (Ripgut Brome); Bromus rubens (Red Brome); Chloris elata (Tall Windmill Grass); Chloris truncata (Windmill Grass); Conyza bonariensis (Hairy Fleabane); Conyza canadensis (Horseweed); Conyza sumatrensis (Sumatran Fleabane); Cynodon hirsutus (Gramilla mansa); Digitaria insularis (Sourgrass); Echinochloa colona (Junglerice); Eleusine indica (Goosegrass); Hedyotis verticillata (Woody borreria); Kochia scoparia (Kochia); Leptochloa virgata (Tropical Sprangletop, Juddsgrass); Lolium perenne (Perennial Ryegrass); Lolium perenne ssp. multiflorum (Italian Ryegrass); Lolium rigidum (Rigid Ryegrass); Parthenium hysterophorus (Ragweed Parthenium); Plantago lanceolata (Buckhorn Plantain); Poa annua (Annual Bluegrass); Raphanus raphanistrum (Wild Radish); Sonchus oleraceus (Annual Sowthistle); Sorghum halepense (Johnsongrass); Urochloa panicoides (Liverseedgrass); and any combination thereof. By reducing resistance or reversing it, a pesticide or herbicide may be used for a longer period of time and/or in lower concentrations or amounts.

Agriculture pests include, but are not limited to, agriculture pest insects, agriculture pest mites, agriculture pest nematodes, grape pests, pest molluscs, strawberry pests, Western honey bee pests, insect pests of ornamental plants, insect vectors of plant pathogens, plant pathogenic nematodes, invasive species, and any combination thereof.

Agriculture pest insects include, but are not limited to, Acalymma, Acrythosiphon kondoi, Acyrthosiphon gossypii, Acyrthosiphon pisum, African armyworm, Africanized bee, Agrilus planipennis (Emerald ash borer), Agromyzidae, Agrotis ipsilon, Agrotis munda, Agrotis porphyricollis, Akkaia taiwana, Aleurocanthus woglumi, Aleyrodes proletella, Alphitobius diaperinus, Alsophila aescularia, Altica chalybea, Ampeloglypter ater, Anasa tristis, Anisoplia austriaca, Anthonomus pomorum, Anthonomus signatus, Aonidiella aurantii, Apamea apamiformis, Apamea niveivenosa, Aphid, Aphis gossypii, Aphis nasturtii, Apple maggot, Argentine ant, Army cutworm, Arotrophora arcuatalis, Astegopteryx bambusae, Astegopteryx insularis, Astegopteryx minuta, Asterolecanium coffeae, Atherigona reversura, Athous haemorrhoidalis, Aulacophora, Aulacorthum solani, Australian plague locust, Bactericera cockerelli, Bactrocera, Bactrocera correcta, Bagrada hilaris, Beet armyworm, Black bean aphid, Blepharidopterus chlorionis, Bogong moth, Boll weevil, Bollworm, Brassica pod midge, Brevicoryne brassicae, Brown locust, Brown marmorated stink bug, Brown planthopper, Cabbage moth, Cabbage worm, Callosobruchus maculatus, Cane beetle, Carrot fly, Cerataphis brasiliensis, Ceratitis capitata, Ceratitis rosa, Ceratoglyphina bambusae, Ceratopemphigus zehntneri, Ceratovacuna lanigera, Cereal leaf beetle, Chaetosiphon tetrarhodum, Chlorops pumilionis, Chrysophtharta bimaculata, Citrus flatid planthopper, Citrus long-horned beetle, Coccus hesperidum, Coccus viridis, Codling moth, Coffee borer beetle, Colorado potato beetle, Confused flour beetle, Crambus, Cucumber beetle, Curculio nucum, Curculio occidentis, Cutworm, Cyclocephala borealis, Date stone beetle, Delia (genus), Delia antiqua, Delia floralis, Delia radicum, Desert locust, Diabrotica, Diabrotica balteata, Diabrotica speciosa, Diamondback moth, Diaphania indica, Diaphania nitidalis, Diaphorina citri, Diaprepes abbreviatus, Diatraea saccharalis, Differential grasshopper, Dociostaurus maroccanus, Drosophila suzukii, Dryocosmus kuriphilus, Dysaphis crataegi, Earias perhuegeli, Epicauta vittata, Epilachna varivestis, Erionota thrax, Eriosoma lanigerum, Eriosomatinae, Euleia heraclei, Eumetopina flavipes, Eupoecilia ambiguella, European corn borer, Eurydema oleracea, Eurygaster integriceps, Ferrisia virgata, Forest bug, Frankliniella tritici, Galleria mellonella, Garden Dart, Geoica lucifuga, Glassy-winged sharpshooter, Great French Wine Blight, Greenhouse whitefly, Greenidea artocarpi, Greenidea formosana, Greenideoida ceyloniae, Gryllotalpa orientalis, Gypsy moths in the United States, Helicoverpa armigera, Helicoverpa gelotopoeon, Helicoverpa punctigera, Helicoverpa zea, Heliothis virescens, Henosepilachna vigintioctopunctata, Hessian fly, Hyalopterus pruni, Hysteroneura setariae, Ipuka dispersum, Jacobiasca formosana, Japanese beetle, Kaltenbachiella elsholtriae, Kaltenbachiella japonica, Khapra beetle, Knulliana, Lampides boeticus, Leaf miner, Leek moth, Lepidiota consobrina, Lepidosaphes beckii, Lepidosaphes ulmi, Leptocybe, Leptoglossus zonatus, Leptopterna dolabrata, Lesser wax moth, Leucoptera (moth), Leucoptera caffeina, Light brown apple moth, Light brown apple moth controversy, Lipaphis erysimi, Lissorhoptrus oryzophilus, Long-tailed skipper, Lygus, Lygus hesperus, Maconellicoccus hirsutus, Macrodactylus subspinosus, Macrosiphoniella pseudoartemisiae, Macrosiphoniella sanborni, Macrosiphum euphorbiae, Maize weevil, Manduca sexta, Matsumuraja capitophoroides, Mayetiola hordei, Mealybug, Megacopta cribraria, Melanaphis sacchari, Micromyzus judenkoi, Micromyzus kalimpongensis, Micromyzus niger, Moth, Myzus ascalonicus, Myzus boehmeriae, Myzus cerasi, Myzus obtusirostris, Myzus ornatus, Myzus persicae, Neomyzus circumflexus, Neotoxoptera oliveri, Nezara viridula, Nomadacris succincta, Oak processionary, Oebalus pugnax, Olive fruit fly, Ophiomyia simplex, Opisina arenosella, Opomyza, Opomyza forum, Opomyzidae, Oscinella frit, Ostrinia furnacalis, Oxycarenus hyalinipennis, Papilio demodocus, Paracoccus marginatus, Paralobesia viteana, Paratachardina pseudolobata, Pentalonia nigronervosa, Pentatomoidea, Phorodon humuli, Phthorimaea operculella, Phyllophaga, Phylloxeridae, Phylloxeroidea, Pieris brassicae, Pink bollworm, Planococcus citri, Platynota idaeusalis, Plum curculio, Prionus californicus, Pseudococcus maritimus, Pseudococcus viburni, Pseudoregma bambucicola, Pyralis farinalis, Red imported fire ant, Red locust, Rhagoletis cerasi, Rhagoletis indifferens, Rhagoletis mendax, Rhodobium porosum, Rhopalosiphoninus latysiphon, Rhopalosiphum maidis, Rhopalosiphum padi, Rhopalosiphum rufiabdominale, Rhyacionia frustrana, Rhynchophorus ferrugineus, Rhynchophorus palmarum, Rhyzopertha, Rice moth, Russian wheat aphid, San Jose scale, Scale insect, Schistocerca americana, Schizaphis graminum, Schizaphis hypersiphonata, Schizaphis minuta, Schizaphis rotundiventris, Schoutedenia lutea, Sciaridae, Scirtothrips dorsalis, Scutelleridae, Scutiphora pedicellata, Serpentine leaf miner, Setaceous Hebrew character, Shivaphis celti, Silver Y, Silverleaf whitefly, Sinomegoura citricola, Sipha flava, Sitobion avenae, Sitobion lambersi, Sitobion leelamaniae, Sitobion miscanthi, Sitobion pauliani, Sitobion phyllanthi, Sitobion wikstroemiae, Small hive beetle, Southwestern corn borer, Soybean aphid, Spodoptera cilium, Spodoptera litura, Spotted cucumber beetle, Squash vine borer, Stemborer, Stenotus binotatus, Strauzia longipennis, Striped flea beetle, Sunn pest, Sweetpotato bug, Synanthedon exitiosa, Tarnished plant bug, Tetraneura nigriabdominalis, Tetraneura yezoensis, Thrips, Thrips angusticeps, Thrips palmi, Tinocallis kahawaluokalani, Toxoptera aurantii, Toxoptera citricida, Toxoptera odinae, Trioza erytreae, Turnip moth, Tuta absoluta, Uroleucon minutum, Varied carpet beetle, Vesiculaphis caricis, Virachola isocrates, Waxworm, Western corn rootworm, Western flower thrips, Wheat fly, Wheat weevil, Whitefly, Winter moth, Xylotrechus quadripes, and any combination thereof.

Agriculture pest mites include, but are not limited to, Abacarus hystrix, Abacarus sacchari, Acarapis woodi, Aceria guerreronis, Aceria tosichella, Brevipalpus phoenicis, Dermanyssus gallinae, Eriophyes padi, Eriophyidae, Flour mite, Oligonychus sacchari, Panonychus ulmi, Polyphagotarsonemus latus, Redberry mite, Steneotarsonemus spinki, Tetranychus urticae, Tuckerella, Varroa destructor, Varroa jacobsoni, Varroa sensitive hygiene, and any combination thereof.

Agriculture pest nematodes include, but are not limited to, Achlysiella williamsi, Anguina (nematode), Anguina agrostis, Anguina amsinckiae, Anguina australis, Anguina balsamophila, Anguina funesta, Anguina graminis, Anguina spermophaga, Anguina tritici, Aphelenchoides, Aphelenchoides arachidis, Aphelenchoides besseyi, Aphelenchoides fragariae, Aphelenchoides parietinus, Aphelenchoides ritzemabosi, Aphelenchoides subtenuis, Belonolaimus, Belonolaimus gracilis, Belonolaimus longicaudatus, Cereal cyst nematode, Coffee root-knot nematode, Ditylenchus, Ditylenchus africanus, Ditylenchus angustus, Ditylenchus destructor, Ditylenchus dipsaci, Dolichodorus heterocephalus, Fig Pin Nematode, Foliar nematode, Globodera pallida, Globodera rostochiensis, Globodera tabacum, Helicotylenchus dihystera, Hemicriconemoides kanayaensis, Hemicriconemoides mangiferae, Hemicycliophora arenaria, Heterodera avenae, Heterodera cajani, Heterodera carotae, Heterodera ciceri, Hoplolaimus galeatus, Hoplolaimus indicus, Hoplolaimus magnistylus, Hoplolaimus seinhorsti, Hoplolaimus uniformis, Longidorus africanus, Longidorus maximus, Longidorus sylphus, Meloidogyne acronea, Meloidogyne arenaria, Meloidogyne artiellia, Meloidogyne brevicauda, Meloidogyne chitwoodi, Meloidogyne enterolobii, Meloidogyne incognita, Meloidogyne javanica, Meloidogyne naasi, Meloidogyne partityla, Meloidogyne thamesi, Merlinius brevidens, Mesocriconema xenoplax, Nacobbus aberrans, Northern root-knot nematode, Paralongidorus maximus, Paratrichodorus minor, Paratylenchus curvitatus, Paratylenchus elachistus, Paratylenchus macrophallus, Paratylenchus microdorus, Paratylenchus projectus, Paratylenchus tenuicaudatus, Potato cyst nematode, Pratylenchus alleni, Quinisulcius acutus, Quinisulcius capitatus, Radopholus similis, Soybean cyst nematode, Tylenchorhynchus, Tylenchorhynchus brevilineatus, Tylenchorhynchus claytoni, Tylenchorhynchus dubius, Tylenchorhynchus maximus, Tylenchorhynchus nudus, Tylenchorhynchus phaseoli, Tylenchorhynchus vulgaris, Tylenchorhynchus zeae, Tylenchulus semipenetrans, Xiphinema, Xiphinema americanum, Xiphinema bakeri, Xiphinema brevicolle, Xiphinema diversicaudatum, Xiphinema insigne, Xiphinema rivesi, Xiphinema vuittenezi, and any combination thereof.

Grape pests include, but are not limited to, Ampeloglypter ater, Ampeloglypter sesostris, Eriophyes vitis, Eupoecilia ambiguella, Fig Pin Nematode, Great French Wine Blight, Japanese beetle, List of Lepidoptera that feed on grapevines, Maconellicoccus hirsutus, Mesocriconema xenoplax, Otiorhynchus cribricollis, Paralobesia viteana, Paratrichodorus minor, Phylloxera, Pseudococcus maritimus, Pseudococcus viburni, Tetranychus urticae, Xiphinema index, Zenophassus, and any combination thereof.

Pest molluscs include, but are not limited to, Cornu aspersum, Deroceras, Grove snail, Limax, Milax gagates, Theba pisana, and any combination thereof.

Strawberry pests include, but are not limited to, Anthonomus rubi, Anthonomus signatus, Aphelenchoides fragariae, Otiorhynchus ovatus, Pratylenchus coffeae, Xiphinema diversicaudatum, and any combination thereof.

Western honey bee pests include, but are not limited to, Acarapis woodi, American foulbrood, Braula, Deformed wing virus, List of diseases of the honey bee, Nosema apis, Small hive beetle, Varroa destructor, Waxworm, and any combination thereof.

Insect pests of ornamental plants include, but are not limited to, Acleris variegana, Acyrthosiphon pisum, Alsophila aescularia, Aphid, Bird-cherry ermine, Coccus hesperidum, Coccus viridis, Contarinia quinquenotata, Grapeleaf skeletonizer, Gypsy moths in the United States, Japanese beetle, Macrodactylus subspinosus, Mealybug, Mullein moth, Orchidophilus, Otiorhynchus sulcatus, Paratachardina pseudolobata, Paysandisia archon, Sawfly, Scale insect, Scarlet lily beetle, Sciaridae, Spodoptera cilium, Stephanitis takeyai, Tenthredo scrophulariae, Yponomeuta malinellus, Yponomeuta padella, and any combination thereof.

Insect vectors of plant pathogens include, but are not limited to, Acyrthosiphon pisum, Agromyzidae, Anthomyiidae, Aphid, Bark beetle, Beet leafhopper, Brevicoryne brassicae, Cacopsylla melanoneura, Chaetosiphon fragaefolii, Cicadulina, Cicadulina mbila, Common brown leafhopper, Cryptococcus fagisuga, Curculionidae, Diabrotica balteata, Empoasca decedens, Eumetopina flavipes, Euscelis plebejus, Frankliniella tritici, Glassy-winged sharpshooter, Haplaxius crudus, Hyalesthes obsoletus, Hylastes ater, Jumping plant louse, Leaf beetle, Leafhopper, Macrosteles quadrilineatus, Mealybug, Melon fly, Molytinae, Pegomya hyoscyami, Pissodes, Pissodes strobi, Pissodini, Planthopper, Pseudococcus maritimus, Pseudococcus viburni, Psylla pyri, Rhabdophaga rosaria, Rhynchophorus palmarum, Scaphoideus titanus, Scirtothrips dorsalis, Silverleaf whitefly, Tephritidae, Thripidae, Thrips palmi, Tomicus piniperda, Toxoptera citricida, Treehopper, Triozidae, Western flower thrips, Xyleborus glabratus, and any combination thereof.

Plant pathogenic nematodes include, but are not limited to, Helicotylenchus, Heterodera, Heterodera amygdali, Heterodera arenaria, Heterodera aucklandica, Heterodera bergeniae, Heterodera bifenestra, Heterodera cacti, Heterodera canadensis, Heterodera cardiolata, Heterodera cruciferae, Heterodera delvii, Heterodera elachista, Heterodera filipjevi, Heterodera gambiensis, Heterodera goettingiana, Heterodera hordecalis, Heterodera humuli, Heterodera latipons, Heterodera medicaginis, Heterodera oryzae, Heterodera oryzicola, Heterodera rosii, Heterodera sacchari, Heterodera schachtii, Heterodera tabacum, Heterodera trifolii, Heteroderidae, Hirschmanniella oryzae, Hoplolaimidae, Hoplolaimus columbus, Hoplolaimus pararobustus, Meloidogyne fruglia, Meloidogyne gajuscus, Nacobbus dorsalis, Pratylenchus brachyurus, Pratylenchus coffeae, Pratylenchus crenatus, Pratylenchus dulscus, Pratylenchus fallax, Pratylenchus flakkensis, Pratylenchus goodeyi, Pratylenchus hexincisus, Pratylenchus loosi, Pratylenchus minutus, Pratylenchus mulchandi, Pratylenchus musicola, Pratylenchus neglectus, Pratylenchus penetrans, Pratylenchus pratensis, Pratylenchus reniformia, Pratylenchus scribneri, Pratylenchus thornei, Pratylenchus vulnus, Pratylenchus zeae, Punctodera chalcoensis, Root gall nematode, Root invasion (parasitic), Root-knot nematode, Rotylenchulus, Rotylenchulus parvus, Rotylenchulus reniformis, Rotylenchus brachyurus, Rotylenchus robustus, Scutellonema brachyurum, Scutellonema cavenessi, Subanguina radicicola, Subanguina wevelli, and any combination thereof.

Invasive species include, but are not limited to, Acacia mearnsii, Achatina fulica, Acridotheres tristis, Aedes albopictus, Anopheles quadrimaculatus, Anoplolepis gracilipes, Anoplophora glabripennis, Aphanomyces astaci, Ardisia elliptica, Arundo donax, Asterias amurensis, Banana bunchy top virus (BBTV), Batrachochytrium dendrobatidis, Bemisia tabaci, Boiga irregularis, Bufo marinus=Rhinella marina, Capra hircus, Carcinus maenas, Caulerpa taxifolia, Cecropia peltata, Cercopagis pengoi, Cervus elaphus, Chromolaena odorata, Cinara cupressi, Cinchona pubescens, Clarias batrachus, Clidemia hirta, Coptotermes formosanus, Corbula amurensis, Cryphonectria parasitica, Cyprinus carpio, Dreissena polymorpha, Eichhornia crassipes, Eleutherodactylus coqui, Eriocheir sinensis, Euglandina rosea, Euphorbia esula, Fallopia japonica=Polygonum cuspidatum, Felis catus, Gambusia affinis, Hedychium gardnerianum, Herpestes javanicus, Hiptage benghalensis, Imperata cylindrica, Lantana camara, Lates niloticus, Leucaena leucocephala, Ligustrum robustum, Linepithema humile, Lymantria dispar, Lythrum salicaria, Macaca fascicularis, Melaleuca quinquenervia, Miconia calvescens, Micropterus salmoides, Mikania micrantha, Mimosa pigra, Mnemiopsis leidyi, Mus musculus, Mustela erminea, Myocastor coypus, Morella faya, Mytilus galloprovincialis, Oncorhynchus mykiss, Ophiostoma ulmi sensu lato, Opuntia stricta, Oreochromis mossambicus, Oryctolagus cuniculus, Pheidole megacephala, Phytophthora cinnamomi, Pinus pinaster, Plasmodium relictum, Platydemus manokwari, Pomacea canaliculata, Prosopis glandulosa, Psidium cattleianum, Pueraria montana var. lobata, Pycnonotus cafer, Rana catesbeiana, Rattus rattus, Rubus ellipticus, Salmo trutta, Salvinia molesta, Schinus terebinthifolius, Sciurus carolinensis, Solenopsis invicta, Spartina anglica, Spathodea campanulata, Sphagneticola trilobata, Sturnus vulgaris, Sus scrofa, Tamarix ramosissima, Trachemys scripta elegans, Trichosurus vulpecula, Trogoderma granarium, Ulex europaeus, Undaria pinnatifida, Vespula vulgaris, Vulpes vulpes, Wasmannia auropunctata, and any combination thereof.

Accelerating genetic manipulations and genome engineering. An active MCR drive may provide faster propagation of a genetic trait compared to passive Mendelian inheritance. A set of copycat cloning vectors may be generated to be used for active genetics into which a transgene may be cloned, targeted for genomic insertion at a desired site, and then homozygosed in the presence of an unlinked source of cas9. For example, FIG. 5 shows the assembly of mutations A-D in four paralogs of a mouse gene to study a specific trait (e.g., CNS function). Using standard genetics, mutant A is crossed with mutant B to recover double heterozygotes, which are then back crossed to each other to recover double homozygotes at a rate of 1/16. This procedure is repeated for mutant C and mutant D. To assemble all four mutations, the AB mutants are crossed with the CD mutants to recover 1/64 quadruple mutant progeny in the fourth generation. Using MCRs or related “copy-cat” elements, mutant A may be crossed with mutant B to produce 100% AB progeny. Mutant C may be crossed with mutant D to produce 100% CD double mutants. The AB double mutant may be crossed with the CD double mutant to recover 100% quadruple mutants in two generations instead of four using standard genetics. This improvement may cut breeding time in half and increase the percentage of double and quadruple mutants to test (e.g., 100% versus 1/64 (1.6%) for the final cross).

Similar methods may be used to generate libraries of model organisms; generate specific strains, breeds, or mutants of a model organism; for one-step mutagenesis schemes to generate scoreable recessive mutant phenotypes in a single generation; facilitate basic genetic manipulations in diverse experimental and agricultural organisms (e.g., accelerating the generation of combinatorial mutants and facilitating mutagenesis in polyploid organisms); accelerate genetic manipulations in animals (e.g., primates) or plants (e.g., trees) with a long generation time; and for gene therapy.

Model organisms include, but are not limited to, viruses, prokaryotes, eukaryotes, protists, fungi, plants, invertebrate animals, vertebrate animals, and any combination thereof. A model organism may include, but is not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

Virus model organisms include, but are not limited to, Phage lambda; Phi X 174; SV40; T4 phage; Tobacco mosaic virus; Herpes simplex virus; and any combination thereof.

Prokaryotic model organisms include, but are not limited to, Escherichia coli; Bacillus subtilis; Caulobacter crescentus; Mycoplasma genitalium; Aliivibrio fischeri; Synechocystis; Pseudomonas fluorescens; and any combination thereof.

Protist model organisms include, but are not limited to, Chlamydomonas reinhardtii; Dictyostelium discoideum; Tetrahymena thermophila; Emiliania huxleyi; Thalassiosira pseudonana; and any combination thereof.

Fungal model organisms include, but are not limited to, Ashbya gossypii; Aspergillus nidulans; Coprinus cinereus; Cryptococcus neoformans; Cunninghamella elegans; Neurospora crassa; Saccharomyces cerevisiae; Schizophyllum commune; Schizosaccharomyces pombe; Ustilago maydis; and any combination thereof.

Plant model organisms include, but are not limited to, Arabidopsis thaliana; Boechera; Selaginella moellendorffii; Brachypodium distachyon; Setaria viridis; Lotus japonicus; Lemna gibba; Maize (Zea mays L.); Medicago truncatula; Mimulus guttatus; Nicotiana benthamiana; Nicotiana tabacum; Rice (Oryza sativa); Physcomitrella patens; Marchantia polymorpha; Populus; and any combination thereof.

Invertebrate animal model organisms include, but are not limited to, Amphimedon queenslandica; Arbacia punctulata; Aplysia; Branchiostoma floridae; Caenorhabditis elegans; Caledia captiva (Orthoptera); Callosobruchus maculatus; Chorthippus parallelus; Ciona intestinalis; Daphnia spp.; Coelopidae; Diopsidae; Drosophila (e.g., Drosophila melanogaster); Euprymna scolopes; Galleria mellonella; Gryllus bimaculatus; Hydra; Loligo pealei; Macrostomum lignano; Mnemiopsis leidyi; Nematostella vectensis; Oikopleura dioica; Oscarella carmela; Parhyale hawaiensis; Platynereis dumerilii; Podisma spp.; Pristionchus pacificus; Scathophaga stercoraria; Schmidtea mediterranea; Stomatogastric ganglion; Strongylocentrotus purpuratus; Symsagittifera roscoffensis; Tribolium castaneum; Trichoplax adhaerens; Tubifex tubifex; and any combination thereof.

Vertebrate animal model organisms include, but are not limited to, Laboratory mice; Bombina bombina, Bombina variegata; Cat (Felis sylvestris catus); Chicken (Gallus gallus domesticus); Cotton rat (Sigmodon hispidus); Dog (Canis lupus familiaris); Golden hamster (Mesocricetus auratus); Guinea pig (Cavia porcellus); Little brown bat (Myotis lucifugus); Medaka (Oryzias latipes, or Japanese ricefish); Mouse (Mus musculus); Poecilia reticulata; Rat (Rattus norvegicus); Rhesus macaque (or Rhesus monkey) (Macaca mulatta); Sea lamprey (Petromyzon marinus); Takifugu (Takifugu rubripes); Xenopus tropicalis; Xenopus laevis; Zebra finch (Taeniopygia guttata); Zebrafish (Danio rerio); African Killifish (Nothobranchius furzeri); Human (Homo sapiens); and any combination thereof.

### EXAMPLES

An MCR construct (y-MCR) targeting the Drosophila yellow (y) locus are generated. Transgenic flies carrying this construct are recovered. The y-MCR construct is transmitted via the germline with an efficiency of 97% indicating that, within the germ cell lineage, MCR is highly efficient at converting the second allele to the sequence of the MCR allele. PCR and DNA sequence analysis of flies carrying the y-MCR construct confirm that MCR flies carry the expected precise insertion of the construct at the cleavage site dictated by the guide RNA.

TABLE 1

| | | | | | | | | | | | | HDR |
|---|---|---|---|---|---|---|---|---|---|---|---|---|
| | | | | | | | | | | | | germline |
| | Sex | Total | | | | | | | | | | conversion |
| F0 | of | F2 off- | F2 | F2 | F2 | F2 | | % ♀ | Tot. | Tot. | % y- | rate |
| progenitor | F1 | spring | y − ♂ | y + ♂ | y − ♀ | y + ♀ | Mosaic ♀ | Mosaic | F2 ♀ | HDR ♀ | MCR ♀ | (%) |
| M3 | f1 | 55 | 30 | 0 | 22 | 0 | 3 | 12 | 25 | 25 | 100 | 100 |
| M3 | f2 | 73 | 39 | 0 | 33 | 0 | 1 | 3 | 34 | 34 | 100 | 100 |
| M3 | f3 | 74 | 35 | 1*‡ | 35 | 2 | 1 | 3 | 38 | 36 | 94.7 | 89 |
| M3 | f4 | 69 | 31 | 1* | 34 | 2 | 1 | 3 | 37 | 35 | 94.6 | 89 |
| M3 | f5 | 66 | 28 | 0 | 33 | 1 | 4 | 11 | 38 | 37 | 97.4 | 95 |
| M3 | f6 | 99 | 51 | 0 | 46 | 1 | 1 | 2 | 48 | 47 | 97.9 | 96 |
| F5 | m1 | 30 | — | 15 | 15 | 0 | 0 | 0 | 15 | 15 | 100 | 100 |
| F5 | m2 | 61 | — | 35 | 25 | 1 | 0 | 0 | 26 | 25 | 96.2 | 92 |
| Total/Ave. | — | 527 | 214 | 52 | 243 | 7 | 11 | 4.2 | 261 | 254 | 97.3 | 94.5 |

Table 1 shows propagation of the y− phenotype among progeny of y-MCR parents. Summary of the genetic transmission of the y− phenotype through two generations carrying the y-MCR construct. Two F0 parents were selected for this analysis, one male (M3) and one female (F5) which when mated to y+ flies gave rise to y− female F1 progeny, and hence were scored as carrying the y-MCR construct. For M3 (who had no male y− F1 progeny as expected), 6 of his 37 y− F1 female progeny (f1-6) were then crossed to y+ males to generate an F2 generation. Female F5 gave rise to 14 y− females and 18 y− males, of which two males (m1, m2) were tested for potential inheritance and propagation of the y-MCR construct by crossing them to y+ females and scoring the F2 generation for the y− phenotype. Female F2 y− progeny were each examined closely for mosaicism. The percent of y-MCR progeny was calculated by dividing the number of y− F2 progeny (including mosaics) by the total number of female progeny. The percent of germline cells that were converted by the MCR construct via HDR (homology directed repair) was estimated in female progeny from F1 crosses by assuming that half would be expected to inherit the MCR element directly by Mendelian segregation and would thus give rise to 100% y− progeny (perhaps with some mosaicism) while the other half would bear a y+ chromosome unless it had been converted in the germline of the F1 parent via HDR. This is likely to be an underestimate of the actual germline conversion rate since some females inheriting the F1 y-MCR allele might not give rise to y− progeny. Indeed, as indicated in the male crosses, where all female progeny would be expected to inherit the MCR construct by simple Mendelian transmission, one y+ female (from m2) was found, suggesting that the y+ allele inherited from the female F1 parent somehow evaded HDR conversion.

Two instances were observed in which male progeny inherited y+ alleles from y-MCR carrying females (asterisks). These alleles may either have escaped MCR conversion altogether or perhaps were the result of non-homologous end-joining repair that generated in frame deletions that carry out y gene function but that are protected from further gRNA directed cleavage. The latter case is strongly suggested by the y+ male derived from the female f3, which sequence analysis revealed carries a single nucleotide change at the gRNA cut site within the y locus resulting a T->I substitution (FIG. 6). This guide-resistant allele is unlikely to be a rare sequence polymorphism, since if it were, it should have resulted in 50% of the F2 offspring being y+.

The sequence of one of the two y+ females derived from the same MCR parent (f3) was analyzed and a combined in-frame deletion (7 nucleotides) and insertion (4 nucleotides) was identified, the net effect of which is the substitution of three amino acids (TVG) with two residues (IY) (FIG. 6). The percent of y− males among total male progeny (2%) is less than that for y+ females (6%) raising the possibility that y+ females consist of both y− (guide-cleaved mutant)/+ and y+ (guide-resistant mutant)/+ genotypes. PCR data for entries indicated in bold text are shown in FIG. 2D. F2 progeny from male m2 (bold text) are shown in FIG. 2E. Text indicates averages of % y-MCR and % HDR germline conversion for all lines tested in this table.

Unless defined otherwise, all technical and scientific terms used herein have the same meaning as commonly understood by one of ordinary skill in the art to which the invention pertains. Other specifically defined terms are to be construed in a manner consistent with the definition provided herein. Although any methods and materials similar or equivalent to those described herein can be used in the practice for testing of the present invention, the preferred materials and methods are described herein. In describing and claiming the present invention, the following terminology will be used.

As used in this specification and the appended claims, the singular forms “a”, “an” and “the” include plural referents unless the content clearly dictates otherwise. Thus, for example, reference to “a construct” includes a combination of two or more nucleic acid constructs, and the like.

Other embodiments and uses are apparent to one skilled in the art in light of the present disclosures. Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-126. (canceled)

127. A method of reversibly introducing a nucleic acid sequence into a genome, the method comprising genomically integrating a mutagenic chain reaction (MCR) element from a MCR construct into a cell or organism, wherein the MCR element comprises:
(a) at least one sequence encoding at least one guide polynucleotide, wherein the at least one sequence encoding at least one guide polynucleotide is genomically integrated in the cell or organism; and
(b) a gene encoding an endonuclease.

128. The method of claim 127, wherein the method is further neutralized using a neutralizing chain reaction.

129. A method of neutralizing a mutagenic chain reaction (MCR) element in a cell or organism, the method comprising genomically integrating a neutralizing chain reaction (NCR) element from an NCR construct into the cell or organism, wherein:
the MCR element comprises:
(a) at least one sequence encoding at least one guide polynucleotide, wherein the at least one sequence encoding at least one guide polynucleotide is genomically integrated in the cell or organism; and
(b) a gene encoding an endonuclease;
the NCR element comprises:
(a) at least one sequence encoding at least one guide polynucleotide directing cleavage within or on both sides of the MCR element; and
(b) at least one sequence encoding at least two guide polynucleotides directing cleavage within or outside of the MCR element or no gene encoding an endonuclease; and
the NCR construct comprises:
(a) the NCR element; and
(b) homology arms flanking the at least one guide polynucleotide that directly abut the endonuclease cut site(s) determined by the at least one guide polynucleotide.

130. The method of claim 129, wherein the endonuclease is a Cas protein.

131. The method of claim 129, wherein the method further comprises deletion of the gene encoding the endonuclease from the genome.

132. The method of claim 129, wherein the method further comprises deletion of the at least one sequence encoding at least one guide polynucleotide in the MCR element from the genome.

133. The method of claim 129, wherein the method further comprises deletion of the MCR element from the genome.

134. The method of claim 129, wherein the at least one sequence encoding at least one guide polynucleotide in the NCR element directs cleavage within the gene encoding the endonuclease.

135. The method of claim 129, wherein the NCR element is genomic ally integrated with an efficiency of at least 50%.

136. The method of claim 129, further comprising generating homozygous mutations in the cell or organism.

137. The method of claim 129, wherein the organism is an animal, human, microorganism, insect, plant, or any combination thereof.

138. The method of claim 129, wherein the cell is from a virus, prokaryote, eukaryote, protist, fungus, invertebrate animal, vertebrate animal, microorganism, pathogen, agriculture pest, or any combination thereof.

139. A construct for neutralizing autocatalytic genome editing occurring due to prior integration of a mutagenic chain reaction (MCR) element, the construct comprising:
(a) at least one sequence encoding at least one guide polynucleotide directing cleavage within or on both sides of the MCR element;
(b) homology arms flanking the at least one guide polynucleotide that directly abut the endonuclease cut site(s) determined by the at least one guide polynucleotide; and
(c) at least one sequence encoding at least two guide polynucleotides directing cleavage within or outside of the MCR element or no gene encoding an endonuclease;
wherein the MCR element comprises:
(a) at least one sequence encoding at least one guide polynucleotide, wherein the at least one sequence encoding at least one guide polynucleotide is genomically integrated in a cell or organism; and
(b) a gene encoding an endonuclease.

140. The construct of claim 139, wherein the guide polynucleotides are guide RNAs.

141. The construct of claim 139, wherein the endonuclease is a Cas protein.

142. A method of genomically integrating a neutralizing chain reaction (NCR) element into a cell or organism, the method comprising:
introducing into the cell or organism an NCR construct comprising:
(a) at least one sequence encoding at least one guide polynucleotide directing cleavage within or on both sides of a mutagenic chain reaction (MCR) element;
(b) homology arms flanking the at least one guide polynucleotide that directly abut the endonuclease cut site(s) determined by the at least one guide polynucleotide; and
(c) at least one sequence encoding at least two guide polynucleotides directing cleavage within or outside of the MCR element or no gene encoding an endonuclease; and
genomically integrating an NCR element comprising:
(a) at least one sequence encoding at least one guide polynucleotide directing cleavage within or on both sides of the MCR element; and
(b) at least one sequence encoding at least two guide polynucleotides directing cleavage within or outside of the MCR element or no gene encoding an endonuclease;
wherein the MCR element comprises:
(a) at least one sequence encoding at least one guide polynucleotide; and
(b) a gene encoding an endonuclease; and
wherein the cell or organism comprises an endonuclease or a gene encoding an endonuclease.

143. The method of claim 142, wherein the NCR construct does not comprise a gene encoding an endonuclease.

144. A method for autocatalytic genome editing, the method comprising genomically integrating a mutagenic chain reaction (MCR) element from an MCR construct into a cell or organism, wherein:
the MCR element comprises:
(a) a gene encoding an endonuclease,
(b) at least one sequence encoding at least one guide polynucleotide, and
(c) an effector cassette; and
the MCR construct comprises:
(d) the MCR element; and
(e) homology arms flanking the MCR element, wherein the homology arms directly abut the endonuclease cut site(s) determined by the at least one guide polynucleotide.

145. The method of claim 144, wherein the endonuclease is a Cas protein.

146. The method of claim 144, wherein the sequence encoding at least one guide polynucleotide is under a control of a separate promoter.

147. The method of claim 144, wherein the MCR construct is injected as a DNA plasmid into a germline of the organism to obtain a transgenic organism.

148. The method of claim 147, wherein homozygous mutations are created wherein the transgenic organism carrying the inserted construct on one copy of a chromosome from which it spreads to another chromosome.

149. The method of claim 144, wherein the MCR construct is introduced into somatic cells in an organism so that the construct can be spread to other cells within that organism.

150. The method of claim 144, wherein the autocatalytic genome editing is used to target suppression of crop pests to those actively attacking a crop of interest.

151. The method of claim 144, wherein the autocatalytic genome editing targets a virus, retrovirus, or another disease independent of the type and stage of disease progression.

152. The method of claim 151, wherein the virus is HIV.

153. The method of claim 144, wherein the autocatalytic genome editing generates scoreable recessive mutant phenotypes in a single generation.

154. A construct for autocatalytic genome editing, the construct comprising:
(a) a gene encoding an endonuclease,
(b) at least one sequence encoding at least one guide polynucleotide,
(c) an effector cassette, and
(d) homology arms flanking the gene, the at least one sequence, and the cassette, wherein the homology arms directly abut the endonuclease cut site(s) determined by the at least one guide polynucleotide.

155. The construct of claim 154, wherein the endonuclease is a Cas protein.
